(19) United States
(12) Patent Application Publication
(10) Pub. No.: US 2006/0269548 A1
(43) Pub. Date: Nov. 30, 2006

(54) Lymphatic endothelial cells materials and methods

(76) Inventors: Kari Alitalo, Helsinki (FI); Taija Makinen, Munich (DE)

Correspondence Address: MARSHALL, GERSTEIN & BORUN LLP, 233 S. WACKER DRIVE, SUITE 6300, SEARS TOWER, CHICAGO, IL, 60606, US

(21) Appl. No.: 10/483,203

(22) PCT Filed: Jul. 12, 2002
(86) PCT No.: PCT/US02/22164

§ 371 Date: Sep. 7, 2004

Related U.S. Application Data
(60) Provisional application No. 60/304,880, filed on Jul. 12, 2001.
(60) Provisional application No. 60/317,610, filed on Sep. 6, 2001.

(51) Int. Cl. A61K 48/00; A61K 39/395; G01N 33/567; C12N 5/08; C12N 15/09

(52) U.S. Cl. 424/144.1; 435/7.21; 435/372; 424/93.21; 435/455

(57) ABSTRACT

The present invention is directed to methods and compositions for isolating lymphatic endothelial cells from a mixed population of cells. More particularly, the inventors have found that certain antibodies that recognize the extracellular domain of VEGFR-3 can be used to specifically isolated lymphatic endothelial cells substantially free of other contaminating non-lymphatic endothelial cells. Methods and compositions for producing such cells and using such cells are described.

### FIELD OF THE INVENTION

The present invention relates generally to materials and methods relating to the isolation of endothelial cells and, cells isolated according to the present invention. More specifically, the present invention is directed to obtaining populations of isolated lymphatic endothelial cells.

### BACKGROUND OF THE INVENTION

The lymphatic system is a complex structure organized in parallel fashion to the circulatory system. In contrast to the circulatory system, which utilizes the heart to pump blood throughout the body, the lymphatic system pumps lymph fluid using the inherent contractility of the lymphatic vessels. The lymphatic vessels are not interconnected in the same manner as the blood vessels, but rather form a set of coordinated structures including the initial lymphatic sinuses [Jeltsch et al., Science, 276:1423-1425 (1997); and Castenholz, A., in Olszewski, W. L. (ed.), Lymph Stasis: Pathophysiology, Diagnosis, and Treatment. CRC Press: Boca Raton, Fla. (1991), pp. 15-42] which drain into the lymphatic capillaries and subsequently to the collecting lymphatics which drain into the lymphatic trunks and the thoracic duct which ultimately drains into the venous circulation. The composition of the channels through which lymph passes is varied [Olszewski, W. L., in Olszewski, W. L. (ed), Lymph Stasis: Pathophysiology, Diagnosis, and Treatment. CRC Press: Boca Raton, Fla. (1991), pp. 235-258; and Kinmonth, J. B., in Kinmonth, J. B. (ed), The Lymphatics: Diseases, Lymphography and Surgery. Edward Arnold Publishers: London, England (1972), pp. 82-86], including the single endothelial layers of the initial lymphatics, the multiple layers of the collecting lymphatics including endothelium, muscular and adventitial layers, and the complex organization of the lymph node. The various organs of the body such as skin, lung, and GI tract have components of the lymphatics with various unique features. [See Ohkuma, M., in Olszewski (1991), supra, at pp. 157-190; Uhley, H. and Leeds, S., in Olszewski (1991), supra, at pp. 191-210; and Barrowman, J. A., in Olszewski (1991), at pp. 221-234).]

Vascular endothelial growth factor (VEGF) is a prime regulator of endothelial cell proliferation, angiogenesis, vasculogenesis and vascular permeability (Ferrara, J Mol Med 77:527-543, 1999). Besides VEGF, the VEGF family of growth factors currently contains five other known members, namely placenta growth factor (PIGF), VEGF-B, VEGF-C, VEGF-D and orf viral VEGF homologs (Eriksson and Alitalo, Curr Top Microbiol Immunol., 237:41-57; 1999). Additional novel VEGF-like heparin binding proteins were recently isolated from snake venom (Gasmi et al., Biochem Biophys Res Commun. 268(1):69-72, 2000; Gasmi et al., Biochim Biophys Acta 1481(1):209-12, 2000; Komori et al., 1999). Disruption of the genes encoding either VEGF or any of the three receptors of the VEGF family, VEGFR-1/Flt1, VEGFR-2/Flk1/KDR or VEGFR-3/Flt4 results in embryonic lethality because of failure of blood vessel development (Dumont et al., Science, 282:946-949, 1998; Fong et al., Nature, 376:66-70, 1995; Shalaby et al., Nature, 376:62-66, 1995). Detailed descriptions of these receptors and their ligands are presented in U.S. Pat. No. 5,776,755; U.S. Pat. No.5,607,918; U.S. Pat. No. 5,840,693; U.S. Pat. No. 5,928,939; U.S. Pat. No. 6,130,071; U.S. Pat. No. 6,221,839; U.S. Pat. No. 6,235,713; U.S. Pat. No. 6,245,530; U.S. patent application Ser. No. 09/427,657 filed Oct. 26, 1999; U.S. patent application Ser. No. 09/534,376 filed Mar. 24, 2000; U.S. Patent Application No. 60/262,476 filed Jan. 17, 2001; as well as PCT Application No. PCT/US98/01973, filed Feb. 2, 1998. Each of these documents is specifically incorporated herein by reference in its entirety. Additional disclosure relating to vascular endothelial growth factors and their receptors may be found in for example, U.S. Pat. No. 6,245,512; U.S. Pat. No. 6,168,778; U.S. Pat. No. 6,100,071; U.S. Pat. No. 6,051,698; U.S. Pat. No. 6,040,157; U.S. Pat. No. 6,020473; and U.S. Pat. No. 6,011,003, each incorporated herein by reference.

VEGFR-2 is considered to be the main signal transducing VEGF receptor for angiogenesis and for mitogenesis of endothelial cells. VEGF induces endothelial cell proliferation, migration and survival via activation of VEGFR-2 and subsequent signal transduction pathways including the MAP (mitogen-activated protein) kinase/ERK (extracellular signal regulated kinase) and the phosphatidylinositol (PI) 3-kinase pathways (for a review, see Petrova et al., Exp. Cell. Res. 253:117-130, 1999; Shibuya et al., In Claesson-Welsh, L (ed.) Vascular growth factors and angiogenesis. Springer Verlag, GmbH&Co., KG, Heidelberg, 237:59-83, 1999). Activation of the p42/p44 MAPK (ERK1/ERK2) cascade is linked in many cells to a proliferation response. In addition, this pathway can lead to increased cell survival by stimulating the transcription of pro-survival genes and by posttranslational modification and inactivation of components of the cell death machinery (Bonni et al., Science, 286:1358-1362, 1999; Gupta et al., Exp. Cell. Res., 247:495-504, 1999). The PI3-kinase pathway was also initially linked to mitogenesis, but several studies have subsequently shown that this pathway has an important function in regulating cell survival by activation of the serine/threonine kinase Akt (protein kinase B) (Datta et al., Genes Dev. 13:2905-2927, 1999). Recent studies have also indicated some crosstalk between the MAPK and PI3-kinase signaling pathways: phosphorylation of Raf by Akt resulted in inhibition of the Raf-MEK (MAP kinase kinase)-ERK pathway (Rommel et al., Science 286:1738-1741, 1999; Zimmermann and Moelling, Science 286:1741-1744, 1999).

Molecular biology has identified at least a few genes and proteins postulated to have roles mediating the growth and/or embryonic development of the lymphatic system. One such gene/protein is the receptor tyrosine kinase designated Flt4 (fmns-like tyrosine kinase 4; also referred to as vascular endothelial cell growth factor receptor 3 or VEGFR-3), cloned from human erythroleukaemia cell and placental cDNA libraries. [See U.S. Pat. No. 5,776,755 and U.S. Pat. no. 6,107,046; Aprelikova et al., Cancer Res., 52: 746-748 (1992); Galland et al., Genomics, 13: 475-478 (1992); Galland et al., Oncogene, 8: 1233-1240 (1993); and Pajusola et al., Cancer Res., 52:5738-5743 (1992), all incorporated herein by reference.] Studies showed that, in mouse embryos, a targeted disruption of the VEGFR-3 gene leads to a failure of the remodeling of the primary vascular network, and death after embryonic day 9.5 [umont et al., Science, 282: 946-949 (1998)]. Additional studies have indicated that certain mutations in VEGFR-3 have an apparent causal role in hereditary lymphedema (PCT Publication No. WO 00/58511). However, VEGFR-3 is not exclusive to lymphatic vessels. VEGFR-3 has an essential role in the development of die embryonic blood vasculature, before the emergence of the lymphatic vessels. However, additional studies indicated that, during further development, the expression of VEGFR-3 becomes restricted mainly to lymphatic vessels [Kaipainen, et al., Proc. Natl. Acad. Sci. USA, 92: 3566-3570 (1995)]. However, VEGFR-3 expression also is observed in neovascular blood vessels of at least some tumors (PCT Publication No. WO 00/21560).

The expression of the VEGFR-3 gene starts during mouse embryonic day 8.5 in developing blood vessels, and VEGFR-3 deficient embryos die at midgestation due to defects in the remodeling of primary vascular networks (Dumont et al., Science, 282:946-949, 1998). However, in adult tissues VEGFR-3 expression occurs mainly in the lymphatic endothelia (Kaipainen, et al., Proc. Natl. Acad. Sci. USA, 92: 3566-3570, 1995; Partanen et al., FASEB J., 14:2087-2096, 2000), and VEGFR-3 ligands VEGF-C and VEGF-D can induce growth of the lymphatic vessels (Jeltsch et al., Science, 276:1423-1425, 1997; Veikkola et al., EMBO J. 20: 1223-1231, 2001). In contrast, blocking of VEGFR-3 signaling by use of a soluble VEGFR-3 protein caused regression of developing lymphatic vessels by inducing endothelial cell apoptosis (Mäkinen et al., Nature Med.,7:199-205, 2001). However, the biochemical signaling pathways activated via VEGFR-3 are less well characterized than those of VEGFR-2, making it difficult to ascertain the mechanism of action of these important regulators of lymphatic endothelial cells function. In the absence of such information, therapeutic and diagnostic implications of dysfunctions of these interactions remain elusive.

Previously, a number of VEGFR-3 antibodies have been described, see for example, U.S. Pat. No. 6,107,046 (incorporated herein by reference). In addition, podoplanin has recently been identified as a specific marker for lymphatic endothelium (Breiteneder-Geleff et al., et al., Am. J. Path., 154(2) 385-394, 1999), and LYVE-1, a homolog of the CD44 glycoprotein is purported to be a lymph-specific receptor for hyaluron (Banerji et al., J. Biol. Chem., 144(4)789-801, 1999). However, despite the availability of these markers, at present, there are no adequate methods of obtaining isolated lymphatic endothelial cells. The study of therapeutic and diagnostic implications of various lymphatic cell disorders would be greatly facilitated if such isolated endothelial cells could be obtained and be made available for molecular studies. Moreover, the availability of such cells would provide useful information about the characteristic features of the lymphatic endothelial cells, thereby facilitating further identification of specific areas for therapeutic intervention.

There are a number of disease states such as hereditary lymphedema, cancer metastases and post-surgical edema, which involve aberration in lymphatic endothelial cells and receptors thereon. The ability to isolate, grow and replace lymphatic endothelial cells would be in a useful palliative intervention, treatment or other ameliorative regimen against such disorders. Such interventions would be particularly useful against injury induced lymphedema, for example. Moreover, the availability of such cells would be particularly amenable to cell-specific treatment regimens thereby greatly reducing undesirable side effects as may be seen in e.g., non-cell specific gene therapy or chemotherapy protocols.

### SUMMARY OF THE INVENTION

The present invention, provides improvements in the ability to manipulate endothelial cells and lymphatic and vascular systems that have numerous practical uses in medicine and molecular biology. More particularly, the present invention provides a method for isolating lymphatic endothelial cells from a biological sample of comprising lymphatic endothelial cells, the method comprising contacting said biological sample with an antibody that preferentially recognizes lymphatic endothelial cells as compared to other endothelial cells, under conditions where the antibody binds lymphatic endothelial cells, and isolating lymphatic endothelial cells bound to said antibody. As used herein the term “antibody” is intended to refer to any antibody agent that specifically binds a target antigen (e.g., lymphatic endothelial cells) or any polypeptide that comprises an antigen binding fragment that specifically recognizes the antigen. More particularly, the antibody is one that is immunologically reactive with an epitope on the extracellular domain of VEGFR-3 that is specific for lymphatic endothelial cells. In the context of the present invention, “preferential” or “specific” means that the antibody binds the target antigen e.g., VEGFR-3 on lymphatic endothelial cells) with greater affinity or avidity than it binds similar antigens on other cells (e.g., VEGFR-3 on blood vascular endothelial cells). This differential binding permits the isolation of one cell type from another.

It should be understood that the biological sample may be from any mammalian organism and may be any tissue or fluid sample that could be expected to contain lymphatic endothelial cells. Particularly preferred is a biological sample is from a human patient. In preferred embodiments, the antibody is immobilized on a solid support and said biological sample is contacted with said support to allow the lymphatic endothelial cells to become bound to said antibody and thereby to the support. In other preferred embodiments, the antibody is labeled with a fluorescent label and said lymphatic endothelial cells are isolated using fluorescence activated cell sorting. In alternative preferred embodiments, the antibody is labeled with a magnetic label and lymphatic endothelial cells are isolated using magnetic activated cell sorting. It is contemplated that the lymphatic endothelial cells in the biological sample may be isolated using immunohistochemistry. Other embodiments contemplate the use of immunochromatography to isolate the lymphatic endothelial cells.

It should be understood that the antibody may be a polyclonal antibody or it may be a monoclonal antibody. In preferred embodiments, the antibody is a binding reagent that comprises an antigen binding fragment of 2E11D11. In other embodiments, the antibody is a derivative of 2E11D11. In still further embodiments, the antibody is a binding reagent that comprises an antigen binding fragment derived from the antigen binding fragment of 2E11D11 which has been mutated or altered to have greater binding specificity for a VEGFR-3 epitope that is specific for lymphatic endothelial cells. In other embodiments, the antibody recognizes the same epitope of VEGFR-3 protein that is recognized by 2E11D11. In particularly preferred embodiments, the antibody is 2E11D11. (deposited as accession 01083129 with European Collection of Cell Cultures, Center for Applied Microbiology and Research, Porton Down Salisbury, U.K.). The production of this antibody is described in U.S. Pat. No. 6,107,046 (incorporated herein by reference). In other preferred embodiments, antibody is an anti-podoplanin. In certain embodiments, antibody is mutant or derivative the anti-podoplanin antibody that has a binding specificity for lymphatic endothelial cells.

Certain aspects of the present invention contemplate, a method of isolating blood vascular endothelial cells from a biological sample comprising microvascular endothelial cells, the method comprising contacting the biological sample with an antibody that preferentially recognizes lymphatic endothelial cells as compared to other endothelial cells, wherein the antibody is an antibody that is immunologically reactive with the extracellular domain of VEGFR-3, and removing the lymphatic endothelial cells that are bound by the antibody from microvascular cells that are not bound to the antibody, wherein the microvascular cells not bound to the antibody comprise a population of blood vascular endothelial cells substantially free of lymphatic endothelial cells.

Anther aspect of the present invention contemplates a lymphatic endothelial cell population isolated according to a method comprising contacting a biological sample comprising lymphatic endothelial cells with an antibody that preferentially binds to lymphatic endothelial cells as compared to other endothelial cells, wherein the antibody is an antibody that is immunologically reactive with the extracellular domain of VEGFR-3, and isolating lymphatic endothelial cells that are bound by the antibody. In preferred embodiments, the biological sample comprises a heterogeneous population of endothelial cells. In other preferred embodiments, the sample of cells is a microvascular endothelial cell population. In particularly preferred embodiments, the lymphatic endothelial cell population is substantially free of contaminating blood vascular endothelial cells. In preferred aspects the method of isolating cells comprises expanding the lymphatic endothelial cells in culture.

A further aspect of the present invention describes a blood vascular endothelial cell population isolated according to a method comprising: contacting a population of microvascular endothelial cells with an antibody that preferentially binds to lymphatic endothelial cells as compared to blood vascular endothelial cells, wherein the antibody is an antibody that is immunologically reactive with the extracellular domain of VEGFR-3, and; removing the lymphatic endothelial cells that are bound by the antibody from microvascular cells that are not bound to the antibody, wherein the microvascular cells not bound to the antibody comprise a population of blood vascular endothelial cells substantially free of lymphatic endothelial cells. In specific embodiments, the blood vascular cell population is produced by a method which further comprises expanding the blood vascular endothelial cell population.

A preferred aspect of the present invention particularly contemplates a lymphatic endothelial cell population substantially free of other contaminating endothelial cells. Another preferred aspect of the invention describes a blood vascular endothelial cell population substantially free of other contaminating endothelial cells.

Another preferred embodiment of the invention relates to a method of obtaining a composition substantially enriched in subpopulation of lymphatic endothelial cells comprising obtaining a source of cells comprising microvascular endothelial cells; contacting the cells with a monoclonal antibody that preferentially binds to lymphatic endothelial cells as compared to other endothelial cells, under conditions to allow the binding of the antibody to lymphatic endothelial cells; separating those cells that are specifically bound by the monoclonal antibody, thereby obtaining a composition substantially enriched in a subpopulation of lymphatic endothelial cells. In preferred aspects antibody is an antibody that is immunologically reactive with the extracellular domain of an antigen expressed on lymphatic endothelial cells. In further preferred embodiments, the antigen is VEGFR-3 The invention also encompasses, in preferred aspects, a composition comprising a substantially enriched subpopulation of lymphatic endothelial cells obtained by such a method. In preferred embodiments, the antibody is an anti-podoplanin. In other preferred embodiments, the antibody is 2E11D11, which preferentially recognizes VEGFR-3 expressed on lymphatic endothelial cells.

Other embodiments contemplate a method of ameliorating a lymphatic endothelial cell disorder comprising targeting lymphatic endothelial cells with a therapeutic agent, wherein the therapeutic agent is targeted to the cells using an antibody that preferentially binds to lymphatic endothelial cells as compared to other endothelial cells, wherein the antibody is an antibody that is immunologically reactive with the extracellular domain of VEGFR-3. In specific embodiments, the disorder is selected from the group consisting of lymphoma, hereditary lymphedema, lymphedemas, lymphangiomas, lymphangiosarcomas, lymphangiomatosis, lymphangiectasis, and cystic hygroma.

The present invention further provides a method of ameliorating a lymphatic disorder, wherein the method comprises ex vivo therapy comprising obtaining a biological sample from the patient in need of the therapy, wherein the biological sample comprises microvascular endothelial cells; contacting the microvascular endothelial cells with an antibody that preferentially binds to lymphatic endothelial cells as compared to other endothelial cells, wherein the antibody is an antibody that is immunologically reactive with the extracellular domain of VEGFR-3; isolating lymphatic endothelial cells that are bound by the antibody, transfecting the lymphatic endothelial cells with an expression construct comprising a nucleic acid encoding a protein operably linked to a promoter, in an amount effective to produce the expression of the protein in the cells; and reintroducing the transfected cells to the patient. The encoded protein can be any protein that one might wish to express in lymphatic endothelial cells (e.g., to treat a disease, palliate the symptoms of a disease, or to permit better diagnosis or imaging)

The present invention also provides a method of promoting the growth of lymphatic endothelial cells in culture comprising obtaining the lymphatic endothelial cells according to a method of the present invention; and stimulating the cells with a VEGFR-3 ligand; wherein stimulating the growth of the cells with the VEGFR-3 ligand promotes the survival of the cells in culture as compared to growth in the absence of the stimulation. In particularly preferred embodiments, the VBGFR-3 ligand is VEGF-C, VEGF-C156S or VEGF-D. The method may further comprise stimulating the cells with a VEGFR-2 ligand. In specific embodiments, it is contemplated that the stimulation of the cells protects the cells from apoptosis. In preferred embodiments, the protection of the cells from apoptosis is mediated through the activation of Akt or p42/MAPK signaling molecules. In preferred embodiments, the stimulation of the cells allows the cells to maintain differentiated endothelial cell characteristics.

Also encompassed by the present invention is a method of selectively modulating lymphatic endothelial cells in a mammalian organism comprising isolating lymphatic endothelial cells from the mammalian organism as described by the present invention, contacting the isolated lymphatic endothelial cells with an agent to modulate the lymphatic endothelial cells; and reintroducing the lymphatic endothelial cells into the organism. In preferred aspects, the contacting step comprises introducing an exogenous polynucleotide into the cells. In other preferred embodiments, the organism has a disorder characterized by a genetic mutation in a gene expressed in lymphatic endothelial cells and the contacting comprises introducing an exogenous polynucleotide into the cells to overcome the effects of the genetic mutation in the gene. In specific embodiments, the disorder is hereditary lymphedema For example, the disorder is hereditary lymphedema characterized by a VEGFR-3 mutation and the treatment comprises introducing a wild-type VEGFR-3 allele.

Another aspect of the invention describes a method for imaging lymphatic endothelial cells in tissue from a vertebrate organism, comprising contacting vertebrate tissue suspected of containing a lymphatic endothelial cells with a composition comprising an antibody that preferentially binds to lymphatic endothelial cells as compared to other endothelial cells, under conditions that allow the binding of the antibody to lymphatic endothelial cells; detecting the antibody bound to the lymphatic endothelial cells in the tissue; and imaging lymphatic endothelial cells in the tissue by identifying lymphatic endothelial cells bound by the antibody, wherein the binding of the lymphatic endothelial cells to the antibody indicates the presence and location of lymphatic endothelial cells in the tissue. More particularly, the tissue comprises human tissue. In specific embodiments, the method further comprises the step of washing the tissue, after the contacting step and before the imaging step, under conditions that remove from the tissue antibody that is not bound to the lymphatic endothelial cells in the tissue. The antibody may be an antibody that is immunologically reactive with the extracellular domain of VEGFR-3. In other embodiments, the antibody is an anti-podoplanin antibody. In preferred embodiments, the antibody further comprises a detectable label covalently bound thereto.

The method may be further defined as comprising contacting the tissue with a second compound that specifically binds to a lymphatic endothelial marker that is substantially absent in blood vascular endothelia; and detecting the second compound bound to cells in the tissue; wherein the imaging step comprises identifying lymphatic vessels labeled with both the antibody and the second compound, wherein lymphatic vessels labeled with both the antibody and the second compound correlate with the presence and location of lymphatic endothelial cells in the tissue. In preferred embodiments, the antibody is an antibody that is immunologically reactive with the extracellular domain of VEGFR-3, and the second compound is an anti-podoplanin antibody.

Also contemplated herein is a method of screening for a disease characterized by a change in lymphatic endothelial cells, comprising obtaining a tissue sample from a vertebrate organism suspected of being in a diseased state characterized by changes in lymphatic endothelial cells; exposing the tissue sample to a composition comprising an antibody that preferentially binds to lymphatic endothelial cells as compared to other endothelial cells, under conditions that allow the binding of the antibody to lymphatic endothelial cells in the organism; washing the tissue sample; and screening for the disease by detecting the presence, quantity, or distribution of the bound antibody in the tissue sample.

Another embodiment contemplates a method for specifically detecting lymphatic endothelial cells in a mammal, comprising administering to the mammal a composition comprising an antibody that preferentially binds to lymphatic endothelial cells as compared to other endothelial cells, under conditions that allow the binding of the antibody to lymphatic endothelial cells, and detecting the antibody bound to lymphatic endothelial cells, thereby detecting lymphatic endothelial cells in the organism. The method may further comprise administering to the mammal a second compound that specifically binds to a lymphatic endothelial cell marker; and wherein the detecting step comprises detection of the antibody and the second compound bound to lymphatic endothelial cells. In all the imaging methods of the invention, it is contemplated that the imaging methods may be used in disorders of lymphatic vessels in determining the presence of the disorder, as well as for monitoring the effects of treatment of the disorder. Such methods may be particularly useful in assessing lymphedema e.g., hereditary lymphedema or injury induced edema and other lymphatic vessel disorders.

Additional features and variations of the invention will be apparent to those skilled in the art from the entirety of this application, and all such features are intended as aspects of the invention. Likewise, features of the invention described herein can be re-combined into additional embodiments that also are intended as aspects of the invention, irrespective of whether the combination of features is specifically mentioned above as an aspect or embodiment of the invention. Also, only such limitations which are described herein as critical to the invention should be viewed as such; variations of the invention lacking limitations which have not been described herein as critical are intended as aspects of the invention.

Aspects of the invention may be summarized by genus, and it should be understood that every individual member of the genus is intended as an individual aspect of the invention.

### BRIEF DESCRIPTION OF THE DRAWING(S)

The following drawings form part of the present specification and are included to further demonstrate aspects of the present invention. The invention may be better understood by reference to one or more of the drawings in combination with the detailed description of the specific embodiments presented herein.

FIG. 1A-FIG. 1C. Analysis of the receptor-specificities of different VEGFs using the Ba/F3 bioassay. Measurement of the viability of Ba/F3 cells expressing the chimeric receptors VEGFR-1/EpoR (FIG. 1A), VEGFR-2/EpoR (FIG. 1B) or VEGFR-3/EpoR (FIG. 1C) in the presence of different VEGFs at indicated concentrations. Cell viability was determined using the MT assay. Data represent the mean values from triplicate assays (mean±A s.d.). FIG. 1D-FIG. 1G. Biosensor analysis of the interaction of VEGF-C (FIG. 1D, FIG. 1E) and VEGF-C1 56S (FIG. 1F, FIG. 1G) with VEGFR-3 (FIG. 1D, FIG. 1F) and VEGFR-2 (FIG. 1E, FIG. 1G). Chimeric receptor proteins were immobilized onto a carboxymethylated dextran surface. Growth factors were injected over the surface at a flow rate of 20 μl/min at the indicated concentrations. The sensorgrams shown have been subtracted with the corresponding signal obtained when the same sample was passed over a blank control channel. Kinetic data derived from the biosensor analysis is shown in Table I.

FIG. 2A-FIG. 2B. VEGFR-2 and VEGFR-3, but not VEGFR-1 activating ligands inhibit apoptosis of serum-deprived HMVE cells. Measurement of the cytoplasmic histone-associated DNA fragments (mono- and oligonucleosomes) in serum-starved MVE cells consisting of two cell populations of blood vascular and lymphatic endothelial cells (FIG. 2A) or in the isolated cell populations after magnetic cell sorting using VEGFR-3 antibodies (FIG. 2B). The enrichment factor of cytoplasmic oligonucleosomes in the apoptotic cells grown for 24 h in serum-free medium (BSA) was chosen as 100 (%). Data represent mean values from three independent experiments (mean±s.d.). The following concentrations of growth factors were used: bFGF 10 ng/ml, P1GF 500 ng/ml, VEGF 50 ng/ml, VEGF-C 100 ng/ml, VEGF-C156S 500 ng/ml, VEGF-D 500 ng/ml, ORFV2-VEGF 500 ng/ml and myelin basic protein (MBP) as an irrelevant control protein 500 ng/ml.

FIG. 3. Quantitation of the Annexin-V positive cells (% of adherent cells) in the podoplanin positive and negative cell populations after 72 hours of serum starvation. Data represent mean values from five counted areas (×400) (mean±s.d.).

FIG. 4. VEGFR-2 or VEGFR-3 stimulation leads to PI3-kinase dependent Akt phosphorylation. VEGF (grey circles), VEGF-C (black boxes) and VEGF-C156S (open triangles) induced phosphorylation of Akt with different kinetics. The data represent quantitations of optical densities of the signals from phosphorylated versus total Akt protein from three independent experiments (mean±s.d.).

FIG. 5. VEGFR-3 mediates endothelial cell migration. The migration of HMVE cells in the presence of different VEGFs in a Boyden chamber assay. VEGF-C156S, but not VEGF stimulated migration was blocked by preincubating VEGF-C156S with ten-molar excess of soluble VEGFR-3 (light grey bars). Data represent mean values from three independent experiments (mean±s.d.). The growth factor concentrations used are: VEGF 10 ng/ml, VEGF-C 500 ng/ml, VEGF-D 500 ng/ml and VEGF-C156S 3 μg/ml.

### DETAILED DESCRIPTION OF THE PREFERRED EMBODIMENTS

In the present invention, the inventors have used VEGF-C and its VEGFR-3 specific mutant (VEGFC156S) to study VEGFR-3 signaling in order to provide a more detailed characterization of this signaling. For the first time, it is demonstrated that primary cultures of human dermal endothelial cells consist of distinct lineages of blood vascular and lymphatic endothelial cells and that the latter can be isolated by using antibodies against VEGFR-3.

In particular, it is shown that VEGFR-3 was expressed specifically on the lymphatic endothelial cells and its stimulation protected these cells from serum-deprivation induced apoptosis and increased cell migration. Moreover, the data presented herein shows that VEGFR-3 can induce PKC dependent p42/p44 MAPK activation and wortmannin-sensitive phosphorylation of Akt. These two important signaling cascades have been associated with cell survival (Bonni et al., Science, 286:1358-1362, 1999; Datta et al., Genes Dev. 13:2905-2927, 1999).

Given the details provided herein, one of skill in the art will now be able to employ molecular markers such as VEGFR-3 for the isolation of lymphatic endothelial cells. Moreover, the present invention teaches that the culture of these cells in the presence of specific growth factors is possible without loss of differentiated properties of these cells. Furthermore, it is demonstrated that specific VEGFR-3 ligands can induce cell migration and protect serum-deprived lymphatic endothelial cells from apoptosis via the activation of two important signaling molecules associated with cell survival, Akt and p42/p44 MAPK. The ability to culture lymphatic endothelial cells should now allow further characterization of the VEGFR-3 signaling pathways as well as the molecular features and gene expression profiles of blood vascular versus lymphatic endothelial cells.

### A. METHODS OF MAKING AND USING ENDOTHELIAL CELL

The inventors have found that microvascular endothelial cells consist of two distinct populations of endothelial cells, namely, lymphatic endothelial cells and blood vascular endothelial cells. The present invention, for the first time provides a method of isolating lymphatic endothelial cells from a mixed population of microvascular endothelial cells. A related implication is the ability to provide isolated blood vascular endothelial cells depleted of lymphatic endothelial cells. The present section provides an overview of the present invention, additional details of various aspects of the invention may be found elsewhere throughout the specification.

Given the teachings of the present invention it will now be possible to cultures both types of endothelial cells. Such cultures will not only be useful in providing insights into cell signaling and function of the endothelial cells but also provide for therapeutic intervention of diseases which involve neovascularization, including for example, angiogenesis, lymphangiogenesis, hereditary lymphedema and the like.

There are numerous commercially available sources of microvascular endothelial cells available to those of skill in the art. Such commercially available sources include for example, Promocell (Heidelberg, Germany; Suppliers of HDMEC, proliferating or cryopreserved microvascular endothelial cells); Cell Applications Inc., (San Diego, Calif., USA, Supplier of CADMEC™, microvascular endothelial cells isolated from normal human neonatal foreskin (or adult skin) capillaries). Additional commercially available sources also will be known to those of skill in the art. In addition to the cells, these sources will generally supply exemplary growth culture conditions to be used in order to maintain the cells in a proliferating state. Thus, cell lines and cultures from commercial sources are a particularly useful starting material for the methods of the instant invention. It is contemplated that any cell culture that comprises microvascular endothelial cells may be used in the present invention. Such a cell culture preferably will only contain microvascular endothelial cells, but it should be understood that cell cultures that contain cells other than just lymphatic and blood vascular endothelial cells also will adequately serve as a starting host cell culture for the present invention, so long as some of the cells of the culture are lymphatic endothelial cells.

In addition to commercially available source, one may which to isolate microvascular endothelial cells from various species, including man. Cells from other species including mice, rats, rabbits, dogs, pigs, horses, and primates also are contemplated. Thus, the invention specifically contemplates the use of primary cell culture and especially, primary human microvascular cell culture. The starting primary cell culture may be one that contains only endothelial cells, but it is likely that when the primary cell culture is initially isolated from the subject, such a cell culture also will contain additional cells such as fibroblasts, smooth muscle cells pericytes and other cells specific for the tissue from which the endothelial cells are being isolated. Such contaminating cells can easily be removed using for example, density gradient centrifugation, immunoabsorption chromatography using specific markers for the cells, fluorescence activated cell screening, magnetic activated cell screening and other cell sorting techniques.

Generally, in the event that there is significant organ specificity of microvascular endothelial cells, the primary microvascular endothelial cell culture should be derived from the tissue involved in the diseases one wishes to study or modulate. Methods for isolating these cells will generally be known to those of skill in the art and will involve, digestion of the given tissue with trypsin and collagenase, aggregation of the microvascular endothelial cells induced by for example, exposure to human plasma, density centrifugation, e.g, Percoll density centrifugation, and ultimately selection and culture of the cells after local digestion with trypsin/EDTA under light microscopy.

The cells of the invention are grown in a medium suitable for the growth of endothelial cells e.g., Ham's F12 medium-10% fetal calf serum (FCS). Once such a culture is generated, one of skill in the art will be able to confirm the presence of microvascular endothelial cell by observing characteristics associated with microvascular endothelial cells, such as, e.g., presence of contact inhibition (i.e., grew in monolayer), and expression of classical endothelial markers, including von Willebrand factor (vWF), platelet endothelial cell adhesion molecule 1(PECAM-1, CD31), and transcripts for the angiotensin converting enzyme (ACE), formation of capillary-like structures, and the like. Elsewhere in the present specification, details for exemplary functional assays for microvascular endothelial cells have been provided.

As indicated above, those of skill in the art will be generally aware of conditions for growing microvascular endothelial cells. In the present invention, culture medium of the cells can be supplement with a variety of growth factors and stimulators. In preferred aspects, the cells may be grown in the presence of stimulators of VEGFR-3 and/or stimulators of VEGFR-2, including but not limited to including VEGF, VEGF-C, VEGF-C156S, VEGF-D and ORFV2-VEGF. These and other related agents are well known to those of skill in the art and are described in further detail elsewhere in the specification.

In preferred uses of the invention, it may be advantageous to grow the isolated endothelial cells in culture for a prolonged period of time. In general, growth of such cells in media is often impeded by apoptosis of the cells. The present invention demonstrates that apoptosis of the cells may be inhibited, slowed, or even prevented, by stimulation of the cells in with stimulators of VEGFR-3 and/or VEGFR-2. In particularly preferred embodiments, the survival of lymphatic endothelial cells in the mixed population of microvascular endothelial cells or indeed isolated cultures of lymphatic endothelial cells substantially free of other endothelial cell contaminants is enhanced or increased by supplementing the media with VEGF-C or VEGF-C156S.

Having grown the endothelial cell culture, the method of the invention provides a method of isolating the lymphatic endothelial cells from the mixed cell population microvascular endothelial cells by using an antibody that preferentially recognizes lymphatic endothelial cells as compared to other endothelial cells. More particularly, the antibody would be one which is immunologically reactive with the extracellular domain of VEGFR-3. In a particularly preferred aspect of the invention, it is demonstrated that the anti-VEGFR-3 antibody 2E11D11 is specific for lymphatic endothelial cells. Another exemplary antibody that is specific for lymphatic endothelial cells is anti-podoplanin antibody. While many of the examples in this specification employ these antibodies, it should be understood that given the teachings of the present invention, additional antibodies may be identified that will serve for the isolation purposes of the present invention. For example, as described below, such additional antibodies may be generated through conventional methods for producing monoclonal antibodies, which methods may use the same epitope recognized by these exemplary antibodies. Alternatively, the additional antibodies may be generated and isolated through phage-display techniques well known those of skill in the art. Yet another alternative would be to generate antibodies related to 2E11D11 or anti-podoplanin antibody be site directed mutagenesis at specific sites of the antibody to generate second generation antibodies that are specific for lymphatic endothelial cell. Methods for producing such antibodies are described in greater detail herein below.

By “specific for lymphatic endothelial cells”, it is meant that this antibody preferentially recognizes lymphatic endothelial cells in a mixed population of endothelial cells and does not recognize or recognizes to a lesser degree endothelial cells from a blood vascular endothelial cell lineage. This differential binding permits isolation of one cell type from the mixed population.

Given that the instant invention shows that microvascular endothelial cells consist generally of a mixed population of blood vascular endothelial cells and lymphatic endothelial cells and that the invention for the first time provides details of how to isolate lymphatic endothelial cells from microvascular endothelial cell culture, such that the endothelial cells are substantially free of other contaminant endothelial cells, e.g., blood vascular endothelial cells, it is understandable that the instant invention also encompasses methods of isolating blood vascular endothelial cells substantially free of other contaminant endothelial cells e.g., lymphatic endothelial cells.

When referring to a population of cells that is “substantially free” of contaminant cells, the instant invention does not mean that the cell culture is required to be completely free of contaminant cells. Rather, it is intended that the majority of the cells of the culture are of the given cell type. For example, in a lymphatic endothelial cell culture substantially free of other contaminant endothelial cells it is expected that at least 51% of the cells are lymphatic endothelial cells. More preferably, at least 60% of the cells are lymphatic endothelial cells. Yet more preferred would be a cell culture comprising at least 70% lymphatic endothelial cells, still more preferred would be 75%, 80%, 85%, 86%, 87%, 88%, 89%, 90% lymphatic endothelial cells. A particularly preferred method of the present invention would be one which isolates lymphatic endothelial cells such that the culture comprises above 90% lymphatic endothelial cells. Obviously, the more purified the culture, the greater the percentage of lymphatic endothelial cells in the culture, most preferred would be cell cultures comprising 95%, 96%, 97%, 98% 99% and of course, 100% lymphatic endothelial cells. Of course, it should be understood that these figures are not intended to be limited to lymphatic endothelial cells and also apply to a substantially purified population of blood vascular endothelial cells. In order to determine the cell type, one of skill in the art may determine the presence of markers specific for any given cell. For example, lymphatic endothelial cells may be identified by the presence of VEGFR-3, the presence of podoplanin as well as other lymphatic cell markers such as LYVE-1. Other VEGFR-3 specific antibodies that may be useful in combination with the above markers include 9D9F9 and 7B3F9 as well as those antibodies described in U.S. Pat. No. 6,107,046. Of course, it should be understood that combinations of markers may be particularly useful. Other assays for determining cell function are described herein and are known to those of skill in the art.

In alternative embodiments, the cell culture produced by the methods of the present invention, be it a substantially purified lymphatic endothelial cell culture or a substantially purified endothelial cell culture may be defined in terms of a minimum amount of contaminating cells. By contaminating cells, it is meant any cell that is not the cell type of which the culture is predominantly composed of. For example, a contaminating cell in a lymphatic endothelial cell culture is any cell that is not a lymphatic endothelial cell. Likewise a contaminating cell in a blood vascular endothelial cell culture is any cell that is not a blood vascular endothelial cell. Examples of contaminating cells of a culture of lymphatic endothelial cells are blood vascular endothelial cells and vice versa, of course other cell types such as for example fibroblasts also will fall into the category of contaminating cells. It is relatively easy to identify contaminating cells, for example by searching for cells possessing specific markers. Thus, in preferred embodiments, the method of the present invention produce cell cultures that contain less than 49% containating cells, more preferably, these cultures contain less than 45%, less than 40%, less than 35%, less than 30%, less than 25%, less than 20% less than 15%, less than 14% contaminating cells, less than 13% contaminating cells, less than 12% contaminating cells, less than 11% contaminating cells or less than 10% contaminating cells. Obviously, the more purified the culture, the less the percentage of contaminating cells that are present in the cells of the culture, most preferred would be cell cultures comprising less than 9% contaminating cells, less than 8% contaminating cells, less than 7% contaminating cells, less than 6% contaminating cells, less than 5% contaminating cells, less than 4% contaminating cells, less than 3% contaminating cells, less than 2% contaminating cells, and of course less than 1% contaminating cells.

In certain aspects of the invention, the present invention contemplates therapeutic and diagnostic methods using the isolated cell populations of the present invention. For example, the methods of the present invention may be used to isolate cells from an individual suspected of having a disorder relating to lymphatic endothelial cells or indeed relating to blood vascular endothelial cells. In diagnostic applications, the cells from the patient individual would be analyzed to determine the presence, absence or alteration of certain cellular or biochemical makers or characteristics of the cells that may be indicative of the diseased state. Similar analyses may be performed for prognostic purposes, in which the cells are isolated before and after the administration of a particular therapy directed at treating the disorder that relates to the cells and determining whether the therapeutic intervention has had a desired effect. In still further embodiments, the isolated cells of the present invention may be used to facilitate an efficacious treatment of a disorder related to an aberration in the physical, biochemical or molecular characteristics of cells. In exemplary embodiments, the therapy may be facilitated by testing the effects of a potential therapy on the cells of the patient in vitro to determine whether the cells of that patient will be responsive to such an intervention. Alternatively, the cells may be used for ex vivo gene therapy in which the cells isolated from a patient are transduced with a genetic expression construct in vitro, expanded and redelivered to the individual in order to correct a disorder in the patient at a molecular level. In yet another alternative, the isolated, expanded cells of the present invention may be used to deliver therapy to a given area. For example, prior to re-delivery of the cells to the patient, the cells are linked to a cytotoxic agent thereby specifically targeting only for example, the lymphatic endothelial cells of the individual. These and other aspects of the invention are discussed in greater detail herein below.

### B. ELUCIDATION OF ROLE OF VEGFR-3 SIGNALING PATHWAYS

Given that the present invention for the first time allows the isolation of a lymphatic endothelial cell population that is substantially free of contaminating cells, it is now possible to determine the elusive role of VEGFR-3 signaling in lymphatic endothelial cells, the role of these cells in phenomena such as lymphangiogensis and in lymphatic disorders.

While stimulation of VEGFR-2 promotes cell viability, VEGF withdrawal results in endothelial cell apoptosis, inhibits angiogenesis and leads to blood vessel regression in vivo (Aiello et al., Proc. Nat'l Acad. Sci., 92: 10457-10461, 1995; Ferrara et al., Nature Med., 4:336-340, 1998; Gerber et al., Development, 126:1149-1159, 1999). Similarly, the inhibition of VEGFR-3 signaling causes regression of developing lymphatic vessels (Mäkinen et al., Nature Med., 7:199-205, 2001). In agreement with previously published studies, VEGFR-2 stimulation strongly protected serum-deprived primary endothelial cells against apoptosis. This effect occurred via VEGFR-2 alone (stimulation by ORFV2-VEGF) as well as in combination with VEGFR-1 stimulation (by VEGF) or VEGFR-3 stimulation (by VEGF-C). However, VEGFR-1 (stimulation by PIGF) transmitted only very weak, if any, cell survival signals. Moreover, VEGFR-3 signaling alone was sufficient for inhibition of serum-deprivation induced apoptosis. Interestingly, while VEGF-C was a weaker survival factor than VEGF for blood vascular endothelial cells, it strongly promoted the survival of VEGFR-3 expressing lymphatic endothelial cells.

VEGFR-3 induced the phosphorylation of two important survival signaling molecules, p42/p44 MAPK and Akt. A PKC inhibitor severely reduced VEGFR-3 mediated p42/p44 MAPK phosphorylation, suggesting that this pathway is mainly transmitted via PKC, not via Ras, similarly to what has been previously shown for VEGFR-2 (Doanes et al., Biochem. Biophys. Res. Commun. 255: 545-548, 1999; Takahashi et al., Oncogene, 18:2221-2230, 1999; Yoshiji et al., Cancer Res., 59:4413-4418, 1999). Such pathway is unique among receptor tyrosine kinases since classically PKC-dependent MAPK activation is thought to be employed mainly by certain seven-transmembrane, G protein-coupled receptors. PKC regulates many endothelial cell processes involved in angiogenesis, including endothelial cell proliferation and migration (Harrington et al., Biochem, Biophys. Res. Commun., 271:499-508, 2000; Harrington et al ., J. Biol. Chem., 272:7390-7397, 1997; Ilan et al., J. Cell Sci., 111:3621-3631, 1998) and inhibition of PKC was able to block tumor neovascularization (Yoshiji et al., Cancer Res., 59:4413-4418, 1999).

Although p42/p44 MAPK activation occurred with a similar kinetics in HMVE cells stimulated by VEGF or VEGF-C, the latter induced a more sustained response. Differences in the duration of activation and in the subcellular distribution of p42/p44 MAPK have been reported to lead to divergent cellular responses (Kaiser et al., Exp. Cell Res., 249:349-358, 1999; Marshall, Cell 80: 179-185, 1995; Pang et al., J. Biol. Chem., 270: 13585-13588, 1995). The differences may result from the fact that only VEGF-C can signal simultaneously via VEGFR-2 and VEGFR-3. However, although VEGF-induced homo- or heterodimeric complexes between VEGFR-1 and VEGFR-2 have been shown to differentially regulate mitogenesis (Rahimi et al., J. Biol. Chem., 275:16986-16992, 2000), we could not detect heterodimer formation by VEGFR-2 and VEGFR-3 in the VEGF-C stimulated cells.

The VEGFR-3 specific mutant form of VEGF-C, VEGF-C156S, proved to be a valuable tool for studies of VEGFR-3 mediated signaling (Joukov et al., EMBO J., 15:290-298, 1998; Veikkola et al., EMBO J., 20:1223-1231, 2001; U.S. Pat. No. 6,130,071). In the biosensor analysis, the affinity of VEGF-C156S to VEGFR-3 was reduced in comparison to the wild type VEGF-C. Moreover, the VEGF-C156S induced maximal VEGFR-3 phosphorylation or p42/p44 MAPK activation were not as strong as for VEGF-C. The reason for this is unclear, but VEGF-C156S may be more unstable than the wild type VEGF-C because one of the eight conserved cysteine residues forming the cystine knot growth factor domain has been changed into a serine residue. However, in a transgenic model VEGF-C156S was as efficient as wild type VEGF-C in promoting lymphangiogenesis (Veikkola et al., EMBO J., 20:1223-1231, 2001). Furthermore, the concentrations used in assays described herein should saturate VEGFR-3. Therefore, since even the highest VEGF-C156S concentrations were not as effective as VEGF-C in protecting cells from apoptosis, a simultaneous activation of both VEGFR-2 and VEGFR-3 may be required for the VEGF-C induced maximal survival of the lymphatic endothelial cells.

### C. ANTIBODIES SPECIFIC FOR LYMPHATIC ENDOTHELIAL CELLS

In the present invention, it is shown that cultured human primary microvascular endothelial cells can be separated into distinct, stable lineages of blood vascular and lymphatic endothelial cells by using certain antibodies against the extracellular domain of VEGFR-3 and both lineages can be expanded in culture. The present section describes the antibodies used for these separation techniques and further describes methods for generating additional antibodies that may be employed in the present invention.

Particularly preferred antibodies of the present invention include for example 2E11D11 (Jussila et al., Cancer Res. 58:1599-1604, 1998; U.S. Pat. No. 6,107,046), and anti-human podoplanin (Breiteneder-Geleff et al., et al., Am. J. Path., 154(2) 385-394, 1999). These antibodies are known to those of skill in the art. Production of antibodies specific for VEGFR-3 (also known as Flt4) is detailed in U.S. Pat. No. 6,107,046, which is specifically incorporated herein by reference.

Given that the present invention teaches that 2E11D11 and anti-podoplanin specifically recognize lymphatic endothelial cells, one of skill in the art will be able to produce additional antibodies that recognize the specific epitope or epitopes recognized by these antibodies. Thus, using the section of VEGFR-3 recognized by 2E11D11, one of skill in the art will be able to produce additional antibodies that recognize lymphatic endothelial cells. Thus, the antibody is one that is preferably immunoreactive with a portion of the VEGFR-3 molecule recognized by 2E11D11, or any other portion of VEGFR-3 that allows the antibody to specifically recognize lymphatic endothelial cells preferentially over any other cell type. By preferentially over any other cell type, it is meant that the antibody will be more reactive with lymphatic endothelial cells than with any other cells including other endothelial cells such as blood vascular endothelial cells.

Moreover, the discovery that the 2E11D11 antibody preferentially recognizes VEGFR-3 expressed on lymphatic endothelial cells over VEGFR-3 expressed on blood vessel endothelial cells demonstrates the feasibility of isolating such antibodies using conventional immunization and screening techniques (see e.g., Harlow and Lane, ANTIBODIES: A LABORATORY MANUAL, Cold Spring Harbor Laboratory, Cold Spring Harbor, N.Y., 1988). A population of VEGFR-3 antibodies can be screened for binding specificity or cross-reactivity against different cell populations described herein.

The antibodies that may be used in the present invention include, but are not limited to, polyclonal, monoclonal, chimeric, single chain, Fab fragments and fragments produced by a Fab expression library, bifunctional/bispecific antibodies, humanized antibodies, CDR-grafted antibodies, human antibodies and antibodies which include portions of CDR sequences specific for VEGFR-3. Neutralizing antibodies, i.e., those which inhibit VEGFR-3 activity also may be useful. In a preferred embodiment, an antibody is a monoclonal antibody. Means for preparing and characterizing antibodies are well known in the art (see, e.g., Harlow and Lane, ANTIBODIES: A LABORATORY MANUAL, Cold Spring Harbor Laboratory, Cold Spring Harbor, N.Y., 1988).

Briefly, a polyclonal antibody is prepared by immunizing an animal with an immunogen comprising a polypeptide of the present invention and collecting antisera from that immunized animal. A wide range of animal species can be used for the production of antisera Typically an animal used for production of anti-antisera is a non-human animal including rabbits, mice, rats, hamsters, goat, sheep, pigs or horses. Because of the relatively large blood volume of rabbits, a rabbit is a preferred choice for production of polyclonal antibodies.

Depending on the host species, various adjuvants may be used to increase immunological response. Such adjuvants include but are not limited to Freund's, mineral gels such as aluminum hydroxide, and surface active substances such as lysolecithin, pluronic polyols, polyanions, peptides, oil emulsions, keyhole limpet hemocyanin, and dinitrophenol. BCG (bacilli Calmette-Guerin) and Corynebacterium parvum are potentially useful human adjuvants.

Antibodies, both polyclonal and monoclonal, specific for isoforms of antigen may be prepared using conventional immunization techniques, as will be generally known to those of skill in the art. As used herein, the term “specific for” is intended to mean that the variable regions of the antibodies recognize and bind an epitope that allows the antibody to specifically and preferentially recognize lymphatic endothelial cells and are capable of distinguishing such an epitopc from other antigens, for example other VEGF receptors or the same receptors expressed on non-lymphatic cells. A composition containing antigenic epitopes such as those recognized by 2E11D11 or anti-podoplanin can be used to immunize one or more experimental animals, such as a rabbit or mouse, which will then proceed to produce specific antibodies against the compounds of the present invention. Polyclonal antisera may be obtained, after allowing time for antibody generation, simply by bleeding the animal and preparing serum samples from the whole blood.

Monoclonal antibodies for use in the invention may be prepared using any technique which provides for the production of antibody molecules by continuous cell lines in culture. These include but are not limited to the hybridoma technique originally described by Koehler and Milstein (Nature 256: 495497, 1975), the human B-cell hybridoma technique (Kosbor et al., Immunol Today 4:72, 1983 ; Cote et al., Proc Natl Acad Sci 80: 2026-2030, 1983) and the EBV-hybridoma technique (Cole et al., Monoclonal Antibodies and Cancer Therapy, Alan R Liss Inc, New York N.Y., pp 77-96, (1985).

When the hybridoma technique is employed, myeloma cell lines may be used. Such cell lines suited for use in hybridoma-producing fusion procedures preferably are non-antibody-producing, have high fusion efficiency, and enzyme deficiencies that render them incapable of growing in certain selective media which support the growth of only the desired fused cells (hybridomas). For example, where the immunized animal is a mouse, one may use P3-X63/Ag8, P3-X63-Ag8.653, NS1/1.Ag 4 1, Sp210-Ag14, FO, NSO/U, MPC-11, MPC11-X45-GTG 1.7 and S194/5XX0 Bul; for rats, one may use R210.RCY3, Y3-Ag 1.2.3, IR983F and 4B210; and U-266, GM1500-GRG2, LICR-LON-HMy2 and UC729-6 are all useful in connection with cell fusions. It should be noted that the hybridomas and cell lines produced by such techniques for producing the monoclonal antibodies are contemplated to be novel compositions of the present invention.

In an exemplary method for generating a polyclonal antisera immunoreactive with the chosen VECFR-3 epitope, 50 μg of VEGFR-3 antigen is emulsified in Freund's Complete Adjuvant for immunization of rabbits. At intervals of, for example, 21 days, 50 μg of epitope are emulsified in Freund's Incomplete Adjuvant for boosts.

To generate monoclonal antibodies, a mouse is injected periodically with recombinant VEGFR-3 against which the antibody is to be raised (e.g., 10-20 μg emulsified in Freund's Complete Adjuvant). The mouse is given a final pre-fusion boost of a VEGFR-3 polypeptide containing the epitope that allows specific recognition of lymphatic endothelial cell in PBS, and four days later the mouse is sacrificed and its spleen removed. The spleen is placed in 10 ml serum-free RPMI 1640, and a single cell suspension is formed by grinding the spleen between the frosted ends of two glass microscope slides submerged in serum-free RPMI 1640, supplemented with 2 mM L-glutamine, 1 mM sodium pyruvate, 100 units/ml penicillin, and 100 μg/ml streptomycin (RPMI) (Gibco, Canada). The cell suspension is filtered through sterile 70-mesh Nitex cell strainer (Becton Dickinson, Parsippany, N.J.), and is washed twice by centrifuging at 200 g for 5 minutes and resuspending the pellet in 20 ml serum-free RPMI. Splenocytes taken from three naive Balb/c mice are prepared in a similar manner and used as a control. NS-1 myeloma cells, kept in log phase in RPMI with 11% fetal bovine serum (FBS) (Hyclone Laboratories, Inc., Logan, Utah) for three days prior to fusion, are centrifuged at 200 g for 5 minutes, and the pellet is washed twice as described in the foregoing paragraph.

1×10⁸ spleen cells are combined with 2.0×10⁷ NS-1 cells and centrifuged, and the supernatant is aspirated. The cell pellet is dislodged by tapping the tube, and 1 ml of 37° C. PEG 1500 (50% in 75 mM Hepes, pH 8.0) (Boehringer Mannheim) is added with stirring over the course of 1 minute, followed by the addition of 7 ml of serum-free RPMI over 7 minutes. An additional 8 ml RPMI is added and the cells are centrifuged at 200 g for 10 minutes. After discarding the supernatant, the pellet is resuspended in 200 ml RPMI containing 15% FBS, 100 μM sodium hypoxanthine, 0.4 μM aminopterin 16 μM thymidine (HAT) (Gibco), 25 units/ml IL-6 (Boehringer Mannheim) and 1.5×10⁶ splenocytes/ml and plated into 10 Corning flat-bottom 96-well tissue culture plates (Corning, Corning N.Y.).

On days 2, 4, and 6, after the fusion, 100 μl of medium is removed from the wells of the fusion plates and replaced with fresh medium. On day 8, the fusion is screened by ELISA, testing for the presence of mouse IgG binding to VEGFR-3 as follows. Immulon 4 plates (Dynatech, Cambridge, Mass.) are coated for 2 hours at 37° C. with 100 ng/well of VEGFR-3 diluted in 25 mM Tris, pH 7.5. The coating solution is aspirated and 200 ul/well of blocking solution (0.5% fish skin gelatin (Sigma) diluted in CMF-PBS) is added and incubated for 30 min. at 37° C. Plates are washed three times with PBS with 0.05% Tween 20 (PBST) and 50 μl culture supernatant is added. After incubation at 37° C. for 30 minutes, and washing as above, 50 μl of horseradish peroxidase conjugated goat anti-mouse IgG(fc) (Jackson ImmunoResearch, West Grove, Penn.) diluted 1:3500 in PBST is added. Plates are incubated as above, washed four times with PBST, and 100 μl substrate, consisting of 1 mg/ml o-phenylene diamine (Sigma) and 0.1 μl/ml 30% H₂O₂ in 100 mM Citrate, pH 4.5, are added. The color reaction is stopped after 5 minutes with the addition of 50 μl of 15% H₂SO₄. A₄₉₀ is read on a plate reader (Dynatech).

Selected fusion wells are cloned twice by dilution into 96-well plates and visual scoring of the number of colonies/well after 5 days. The monoclonal antibodies produced by hybridomas are isotyped using the Isostrip system (Boehringer Mannheim, Indianapolis, Ind.).

In addition to the production of monoclonal antibodies, techniques developed for the production of “chimeric antibodies”, the splicing of mouse antibody genes to human antibody genes to obtain a molecule with appropriate antigen specificity and biological activity can be used (Morrison et al., Proc Natl Acad Sci 81: 6851-6855, 1984; Neuberger et al., Nature 312: 604-608, 1984; Takeda et al., Nature 314: 452454; 1985). Alternatively, techniques described for the production of single chain antibodies (U.S. Pat. No. 4,946,778) can be adapted to produce VEGFR-3-specific single chain antibodies.

From an antibody population that is shown to bind VEGFR-3 or other lymphatic endothelial cell antigens, one can use blood vessel endothelial cells to “subtract” those antibodies that cross-react with VEGFR-3 or other epitopes on such cells. The remaining antibody population is enriched in antibodies preferential for lymphatic endothelial cell epitopes.

Antibodies may also be produced by inducing in vivo production in the lymphocyte population or by screening recombinant immunoglobulin libraries or panels of highly specific binding reagents as disclosed in Orlandi et al (Proc Natl Acad Sci 86: 3833-3837; 1989), and Winter G and Milstein C (Nature 349: 293-299, 1991).

It is proposed that the antibodies of the present invention, in addition to being used for the isolation methods of the invention will find useful application in standard immunochemical procedures, such as ELISA and Western blot methods and in immunohistochemical procedures such as tissue staining, as well as in other procedures which may utilize antibodies specific to VEGFR-3-related antigen epitopes.

In general, both polyclonal and monoclonal antibodies made for the present invention may be used in a variety of other embodiments. In certain aspects, the antibodies may be employed for therapeutic purposes in which the inhibition of VEGFR-3 activity is desired. Antibodies may be used to block VEGF-C action and VEGFR-3 receptor function thereby treating hyperproliferative disorders associated with lymphangiogenesis. Antibodies of the present invention also may prove useful in diagnostic purposes in order, for example, to detect increases or decreases in VEGFR-3 proteins in tissue samples including samples for sites of a suspected diseased state. Additional aspects will employ the antibodies of the present invention in antibody cloning protocols to obtain cDNAs or genes encoding other VEGFR-3 proteins. They may also be used in inhibition studies to analyze the effects of VEGFR-3 related peptides in cells or animals. The antibodies produced for the present invention will also be useful in immunolocalization studies to analyze the distribution of VEGFR-3 during various cellular events, for example, to determine the cellular or tissue-specific distribution of VEGFR-3 polypeptides under different points in the cell cycle. A particularly useful application of such antibodies is in purifying native or recombinant VEGFR-3, for example, using an antibody affinity column. The operation of all such immunological techniques will be known to those of skill in the art in light of the present disclosure.

In addition to the above “conventional” methods of generating antibodies for use in the invention, also contemplated are phage display methods of screening for antibodies that would be useful herein. It is now known that 2E11D11 and anti-podoplanin antibodies will specifically recognize lymphatic endothelial cells. Cells isolated by use of these two antibodies can be used as a read-out for other related antibodies that are presented using phage display of all possible mutations of the 2E11D11 or anti-podoplanin related molecules. Alternatively, the displayed antibodies may be selected by their binding capacity to a given epitope recognized by 2E11D11 or anti-podoplanin. This method for isolating novel antibodies is well known to those of skill in the art and detailed in for example, U.S. Pat. No. 5,223,409, incorporated herein by reference, which describes the directed evolution of binding proteins. Related methods also are described in U.S. Pat. No. 5,403,484; U.S. Pat. No. 5,571,698; U.S. Pat. No. 5,837,500; U.S. Pat. No. 5,702,892; . The techniques described in U.S. Pat. No. 5,780,279; U.S. Pat. No. 5,821,047; U.S. Pat. No. 5,824,520; U.S. Pat. No. 5,855,885; U.S. Pat. No. 5,858,657; U.S. Pat. No. 5,871,907; U.S. Pat. No. 5,969,108; U.S. Pat. No. 6,057,098; U.S. Pat. No. 6,225,447, also will be useful for generating antibodies for the present invention.

Additionally, another useful technique for generating antibodies for use in the present invention may be one which uses a rational design type approach. The goal of rational design is to produce structural analogs of biologically active polypeptides or compounds with which they interact (agonists, antagonists, inhibitors, peptidomimetics, binding partners, etc.). In this case, the active polypeptides are 2E11D11 and ant-podoplanin antibodies discussed herein throughout. By creating such analogs, it is possible to fashion additional antibodies which are more immunoreactive than the native or natural 2E11D11 or anti-podoplanin molecules. In one approach, one would generate a three-dimensional structure for the antibodies or an epitope binding fragment thereof This could be accomplished by x-ray crystallograph, computer modeling or by a combination of both approaches. An alternative approach, “alanine scan,” involves the random replacement of residues throughout molecule with alanine, and the resulting affect on function determined.

It also is possible to solve the crystal structure of the specific antibodies. In principle, this approach yields a pharmacore upon which subsequent drug design can be based. It is possible to bypass protein crystallograph altogether by generating anti-idiotypic antibodies to a functional, pharmacologically active antibody. As a mirror image of a mirror image, the binding site of anti-idiotype would be expected to be an analog of the original antigen. The anti-idiotype could then be used to identify and isolate additional antibodies from banks of chemically- or biologically-produced peptides.

### D. METHODS FOR ISOLATING CELLS

The present invention provides a method of isolating lymphatic endothelial cells from a mixed cell culture. Essentially, this isolation method employs antibodies that preferentially recognize lymphatic endothelial cells. The generation and examples of such antibodies have been discussed above. The present section described certain techniques that may be used in conjunction with the antibodies to isolate the cells. These are merely exemplary techniques, those of skill in the art will be aware of other methods for isolating cells that may also be used in combination with the methods described herein.

Various techniques may be employed to separate the cells according to the present invention. The antibodies may be attached to a solid support to allow for crude separation. The separation techniques employed should maximize the retention of viability of the fraction to be collected. Various techniques of different efficacy may be employed to obtain “relatively crude” separations. Such separations are where up to 10%, usually not more than about 5%, preferably not more than about 1%, of the total cells present not having the marker may remain with the cell population to be retained. The particular technique employed will depend upon efficiency of separation, associated cytotoxicity, ease and speed of performance, and necessity for sophisticated equipment and/or technical skill.

Procedures for separation may include, but are not limited to, magnetic separation, using antibody-coated magnetic beads, affinity chromatography, cytotoxic agents joined to a monoclonal antibody or used in conjunction with a monoclonal antibody, including, but not limited to, complement and cytotoxins, and “panning” with antibody attached to a solid matrix, e.g., plate, elutriation or any other convenient technique.

The use of separation techniques include, but are not limited to, those based on differences in physical (density gradient centrifugation and counter-flow centrifugal elutriation), cell surface (lectin and antibody affinity), and vital staining properties (mitochondria-binding dye rho123 and DNA-binding dye Hoechst 33342).

Techniques providing accurate separation include, but are not limited to, Fluorescent Activated Cell Sorting (FACS) FACS, which can have varying degrees of sophistication, e.g., a plurality of color channels, low angle and obtuse light scattering detecting channels, impedance channels, etc.

FACS is a cell sorting method with which cells in suspension can be separated based on differences in cell surface markers. In the context of the present invention, FACS may be used to specifically remove lymphatic endothelial cells from a mixed population of cells. FACS physically separates a cell or particle of interest from a heterogeneous population. Using this techniques, cells can be sorted in a sterile environment enabling the recovered cells to be cultured. The lymphatic endothelial cells are sorted according to the presence of an antigen recognized by the antibodies described herein. Additionally, the FACS also may be used to remove contaminant cells from the cell culture by recognition of antigen expression, GFP expression, DNA content or cell function (e.g. calcium flux or apoptosis) of the contaminant cells. The contaminant cells may be removed before, after or both before and after the lymphatic endothelial cells are isolated.

FACS is based on flow cytometry, which is a means of measuring certain physical and chemical characteristics of cells or particles as they travel in suspension one by one past a sensing point. Thus, flow cytometers can be considered to be specialized fluorescence microscopes. The modern flow cytometer consists of a light source, collection optics, electronics and a computer to translate signals to data In most modern cytometers the light source of choice is a laser which emits coherent light at a specified wavelength. Scattered and emitted fluorescent light is collected by two lenses (one set in front of the light source and one set at right angles) and by a series of optics, beam splitters and filters, specific bands of fluorescence can be measured. Physical characteristics measurable by flow cytometric techniques include characteristics such as cell size, shape and internal complexity and, of course, any cell component or function that can be detected by a fluorescent compound can be examined.

In general, flow cytometers use a principle involving the electrostatic deflection of charged droplets. Cells are aspirated from a sample and ejected one by one from a nozzle in a stream of sheath fluid which is generally PBS but can be any ionized fluid. As the cell intercepts with the laser beam, scattered light and fluorescence signals are generated and the sort logic boards make a decision as to whether the cell is to be sorted or not (according to user-defined criteria). In this instance, the user defined criteria for sorting lymphatic endothelial cells is whether or not the cells bind or are recognized by a lymphatic endothelial cell specific antibody, such as 2E11D11, anti-podoplanin and the like.

The distance between the laser intercept and the break-off point is called the drop delay. If a cell of interest i.e. one to be sorted, has been detected, the cytometer waits until that cells has traveled from the intercept to the break-off point and then charges the stream. So as the drop containing the cell of interest leaves the solid fluid stream it will carry a charge, either positive or negative. A further distance downstream the charged drop passes through two high voltage deflection plates and will be attracted to towards the plate of opposite polarity. So it is possible to sort two separate populations from the same sample.

In a first separation, typically starting with about 1×10⁸⁻⁹ preferably at about 5×10⁸⁻⁹ cells, the lymphatic cell specific antibody may be labeled with one fluorochrome, while the antibodies for the various other cells, or anti-gp80 antibodies, may be conjugated to at least one different fluorochrome. While each of the lineages may be separated in a separate step, desirably the lineages are separated at the same time as one is positively selecting for the epitope recognized by 2E11D11 and/or other lymphatic endothelial markers. The cells may be selected against dead cells, by employing dyes associated with dead cells (including but not limited to, propidium iodide (PI)). Preferably, the cells are collected in a medium appropriate for the growth or storage of the cells. Cells may be selected based on light-scatter properties as well as their expression of various cell surface antigens. Those of skill in the art are well aware of specific protocols that may be used for FACS sorting of the cells for the present invention.

In an exemplary FACS procedure, microvascular endothelial cells are labeled in suspension by incubating with one or more antibody that recognizes the lymphatic endothelial cells at 4° C. for 40 minutes. Cells before and after sorting are maintained at 4° C. and in an appropriate medium. After completion of the antibody labeling, propidium iodide (for identifying dead cells) at final concentration of 10 μg/ml was added to each of the sample tubes. Fluorescence Activated Cell Sorting is performed with a Becton Dickinson FACSTAR^{plus} (San Jose, Calif.) using a 4 W argon laser with 60 mW of power and a 100 μm nozzle. FACS also can be used to measure physical characteristic by determining FSC and SSC scattering of the cells.

In addition to FACS, MACS also is a useful technique for sorting cells. Instead of using immunofluorescence as a method for isolating the cells, the cells are immunomagnetically labeled and separated using magnetic field. Antibody attached to magnetic beads can also be used to separate the lymphatic endothelial cells from a mixed population culture. The magnetic beads presenting the antibody are bound in a column held in a magnetic field. The microvascular cell population is then passed through the column, and the lymphatic endothelial cells become bound by the antibody whereas the remainder of the cells are collected in the column flowthrough.

Conventional immunosorbant affinity chromatography also is contemplated for isolating the lymphatic cells. In such a technique, the antibody is bound to inert column chromatography beads and the beads are packed into a column. When the microvascular cell population is passed through the column, the lymphatic endothelial cells become bound to the antibody whereas the non-lymphatic endothelial cells pass through in the column flowthrough.

Panning techniques also may be used to isolate the lymphatic endothelial cells of the invention. Panning for cells is a well known technique which employs antibodies to bind cells to a solid support such as a petri dish. Essentially, an antibody specific for the cells to be panned for, e.g., 2E11D11 specific for lymphatic endothelial cells is coated onto an adherent cell culture plate. The mixed population of cells is then added to the plate and the plate is swirled in order to allow the cells to come into full contact with the antibody immobilized on the surface of the plate. The remaining cell culture media containing cells that are not recognized by the antibody is removed from the plate, leaving the lymphatic endothelial cells substantially free of contaminating cells, attached to the antibody. The cells can either be harvested and transferred into fresh culture medium for expansion or alternatively, the fresh culture media maybe added to the cells attached to the antibodies. In the case of a microvascular cell population, it should be understood that the cells that remained in suspension and were removed from the adhered lymphatic cells are a population of blood vascular endothelial cells substantially free of contaminating lymphatic endothelial cells.

In an exemplary panning protocol, antibodies, (0.5 mg/dish) diluted in 9 ml of an appropriate buffer are poured onto 100 mm² bacteriological polystyrene petri dishes (Falcon, Lincoln Park, N.J.). The dishes are swirled to evenly coat the surface and incubated at room temperature for 40 minutes. The coated dishes are washed with the buffer prior to use, to remove any residual antibody that has not adhered to the surface of the petri dish. A volume e.g., 10 milliliters of a microvascular cell suspension containing up to 3×10⁷ cells is incubated at 4° C. for 10 minutes in the dishes coated with the antibody. The non-adherent cells are removed by aspiration and the plates are washed with a buffer or media suitable for the cells. The non-adherent cells can be precipitated using centrifugation and recultured.

### E. ASSAYS FOR DETERMINING THE PRESENCE OF VEGFR-3 ACTIVITY

The many biological activities mediated through the VEGF-C/D binding to VEGFR-3 receptor family (including but not limited to affecting growth and migration of vascular endothelial cells and blood vessels; promoting growth of lymphatic endothelial cells and lymphatic vessels; increasing vascular permeability; and affecting myelopoiesis) support numerous in vitro and in vivo clinical utilities for the isolated cells of the present invention. For the first time, such activities can specifically be monitored in lymphatic endothelial cell cultures free of any contaminating effects that may have been caused by non-lymphatic endothelial cells. These cells can be monitored for VEGFR-3 binding and activity of VEGF-C/D as well as compounds that modulate the binding. As such, the cells of the invention will be effective in identifying modulators and in preferred embodiments inhibitors of VEGF-C/D mediated biological responses. The present section describes various assays for determining the presence of VEGFR-3 binding and/or activity. The presence of such activity in the cells isolated by the present invention will be used to indicate that such cells are lymphatic endothelial cells.

The presence of lymphatic endothelial cells may be monitored by the ability of the cells to present VEGFR-3 binding activity. Exemplary binding assays have been described in Achen et al., Proc Natl Acad Sci USA 95:548-53 (1998), incorporated by reference in its entirety. These assays will generally comprise admixing the cells of the present invention which should express the VEGFR-3 receptor and a ligand of the receptor (e.g., VEGF-C) and determining the receptor binding.

The cells may be used for applications where the therapeutic efficacy of an agent in inhibiting VEGFR-3 receptors is desired to be determined prior to administering the agent to the individual. As an indicator of activity, the ability of the therapeutic agent to alter autophosphorylation of VEGFR-3 receptor on the cells can also be examined. A candidate therapeutic agent is added to cells, the cells are then lysed and immuoprecipitated with anti-VEGF receptor antiserum and analyzed by Western blotting using anti-phosphotyrosine antibodies to determine phosphorylation of the VEGF receptor.

The cells in these assays are grown using techniques well known to those of skill in the art. For example, the cells are grown in Ham's F12 medium-10% fetal calf serum (FCS). The cells are starved overnight in DMEM medium or Ham's F12 supplemented with 0.2% bovine serum albumin (BSA), and then incubated for 5 minutes with VEGF-C alone or the therapeutic agent in combination with VEGF-C.

After addition of the VEGF-C, the cells are washed twice with ice-cold Tris-Buffered Saline (TBS) containing 100 mM sodium orthovanadate and lysed in RIPA buffer containing 1 mM phenylmethylsulfonyl fluoride (PMSF), 0.1 U/ml aprotinin and 1 mM sodium orthovanadate. The lysates are sonicated, clarified by centrifugation at 16,000×g for 20 minutes and incubated for 3-6 hours on ice with 3-5 μl of antisera specific for VEGFR-3 or VEGFR-2. Immunoprecipitates are bound to protein A-Sepharose, washed three times with RIPA buffer containing 1 mM PMSF, 1 mM sodium orthovanadate, washed twice with 10 mM Tris-HCl (pH 7.4), and subjected to SDS-PAGE using a 7% gel. Polypeptides are transferred to nitrocellulose by Western blotting and analyzed using PY20 phosphotyrosine-specific monoclonal antibodies (Transduction Laboratories) or receptor-specific antiserum and the ECL detection method (Amersham Corp.).

The ability of a candidate therapeutic to affect the autophosphorylation (detected using the anti-phosphotyrosine antibodies) is scored as modulating the receptor. The level of alteration observed for various concentrations of therapeutic agent, relative to known concentrations of VEGF-C, provide an indication of the potency of receptor modulation. Therapeutics that have been shown to bind the receptor, but are incapable of stimulating receptor phosphorylation, are scored as inhibitors. Inhibitory activity can be further assayed by mixing a known receptor agonist such as recombinant VEGF-C with either media alone or with concentrated conditioned media, to determine if the concentrated conditioned media inhibits VEGF-C-mediated receptor phosphorylation.

The presence of lymphatic endothelial cells can also be monitored using binding assays for natural or recombinant ligands of VEGFR-3. To measure the binding affinities of selected ligands, an ELISA-type approach may be employed. For example, to examine binding affinity for VEGFR-3, serial dilutions of competing VEGFR-3-IgG fusion proteins and a subsaturating concentration of the candidate ligand tagged with the myc epitope is added to microtitre plates coated with VEGFR-3, and incubated until equilibrium is established. The plates are then washed to remove unbound proteins. Ligands that remain bound to the VEGFR-3 coated plates are detected using an anti-myc antibody conjugated to a readily detectable label e.g., horseradish peroxidase. Binding affinities (EC₅₀) can be calculated as the concentration of competing VEGFR-IgG fusion protein that results in half-maximal binding.

VEGF-C stimulates endothelial cell migration in collagen gel. The cells of the invention may be examined to determine VEGF-C mediated endothelial cell migration in collagen gel, thus providing another indicia that the isolated cells are indeed lymphatic endothelial cells. Exemplary cell migration assays have been described in International Patent Publication No. WO 98/33917, incorporated herein by reference. Briefly, the lymphatic endothelial cells isolated in the invention are seeded on top of a collagen layer in tissue culture plates. VEGF-C is placed in wells made in collagen gel approximately 4 mm away from the location of the attached lymphatic endothelial cells. The number of endothelial cells that have migrated from the original area of attachment in the collagen gel towards the wells containing the VEGF-C is then counted to assess VEGF-C induced cell migration.

Collagen gels for these assays are prepared by mixing type I collagen stock solution (5 mg/ml in 1 mM HCl) with an equal volume of 2× MEM and 2 volumes of MEM containing 10% newborn calf serum to give a final collagen concentration of 1.25 mg/ml. Tissue culture plates (5 cm diameter) are coated with about 1 mm thick layer of the solution, which is allowed to polymerize at 37° C. The lymphatic endothelial cells of the invention are seeded atop this layer.

For the migration assays, the cells are allowed to attach inside a plastic ring (1 cm diameter) placed on top of the first collagen layer. After 30 minutes, the ring is removed and unattached cells are rinsed away. A second layer of collagen and a layer of growth medium (5% newborn calf serum (NCS)), solidified by 0.75% low melting point agar (FMC BioProducts, Rockland, Me.), are added. A well (3 mm diameter) is punched through all the layers on both sides of the cell spot at a distance of 4 mm, and media containing a VEGF-C polypeptide is pipetted daily into the wells. Photomicrographs of the cells migrating out from the spot edge are taken, e.g., after six days, through an Olympus CK 2 inverted microscope equipped with phase-contrast optics. The migrating cells are counted after nuclear staining with the fluorescent dye bisbenzimide (1 mg/ml, Hoechst 33258, Sigma).

The number of cells migrating at different distances from the original area of attachment towards wells containing the VEGF-C, are determined 6 days after addition of the media. The number of cells migrating out from the original ring of attachment are counted in five adjacent 0.5 mm×0.5 mm squares using a microscope ocular lens grid and 10× magnification with a fluorescence microscope. Cells migrating further than 0.5 mm are counted in a similar way by moving the grid in 0.5 mm steps. The ability of the cells of the present invention to undergo VEGF-C mediated migration indicates that the cells isolated are lymphatic endothelial cells that express VEGFR-3.

Additionally, the mitogenic activity of VEGF-C can be examined using endothelial cell proliferation assays such as that described in Breier et al., Dev 114:521-532 (1992), incorporated herein in its entirety. The cells may be assayed for this effect by adding the VEGF-C to the cells. After three days, the cells are dissociated with trypsin and counted using a cytometer to determine any effects of the peptides on the proliferative activity of the lymphatic endothelial cells.

### F. STIMULATORS OF VEGFR-3

As indicated in this specification, it is contemplated that the isolated lymphatic endothelial cells may be grown in culture in such a manner that their survival in culture is promoted. In specific embodiments, the cells may be grown in the presence of stimulators of VEGFR-3 and/or stimulators of VEGFR-2, including but not limited to including VEGF, VEGF-C, VEGF-C156S, VEGF-D and ORFV2-VEGF. Certain of these stimulators and their effects on VEGF receptors is discussed in further detail in the present section. It should be understood that these agents may be prepared in any formulation that makes them amenable to use in conjunction with the cells of the present invention. Additionally, these stimulators may be supplied to the cells of the invention either alone or in a combined application to inhibit, suppress, reduce or otherwise prevent apoptosis of the lymphatic endothelial cells in culture.

The above stimulators belong to the PDGF/VEGF family of growth factors, which includes at least the following members: PDGF-A (see e.g., GenBank Acc. No. X06374), PDGF-B (see e.g., GenBank Acc. No. M12783), VEGF (see e.g., GenBank Acc. No. Q16889 referred to herein for clarity as VEGF-A or by particular isoform), PIGF (see e.g., GenBank Acc. No. X54936 placental growth factor), VEGF-B (see e.g., GenBank Acc. No. U48801; also known as VEGF-related factor (VRF)), VEGF-C (see e.g., GenBank Acc. No. X94216; also known as VEGF related protein (VRP)), VEGF-D (also known as c-fos-induced growth factor (FIGF); see e.g., Genbank Acc. No. AJ000185), VEGF-E (also known as NZ7 VEGF or OV NZ7; see e.g., GenBank Acc. No. S67522), NZ2 VEGF (also known as OV NZ2; see e.g., GenBank Acc. No. S67520), D1701 VEGF-like protein (see e.g., GenBank Acc. No. AF106020; Meyer et al., EMBO J 18:363-374), and NZ10 VEGF-like protein (described in International Patent Application PCT/US99/25869) [Stacker and Achen, Growth Factors 17:1-11 (1999); Neufeld et al., FASEB J 13:9-22 (1999); Ferrara, J Mol Med 77:527-543 (1999)].

VEGF-C, comprises a VHD that is approximately 30% identical at the amino acid level to VEGF-A. VEGF-C is originally expressed as a larger precursor protein, prepro-VEGF-C, having extensive amino- and carboxy-terminal peptide sequences flanking the VHD, with the C-terminal peptide containing tandemly repeated cysteine residues in a motif typical of Balbiani ring 3 protein. Prepro-VEGF-C undergoes extensive proteolytic maturation involving the successive cleavage of a signal peptide, the C-terminal pro-peptide, and the N-terminal pro-peptide. Secreted VEGF-C protein consists of a non-covalently-linked homodimer, in which each monomer contains the VHD. The intermediate forms of VEGF-C produced by partial proteolytic processing show increasing affinity for the VEGFR-3 receptor, and the mature protein is also able to bind to the VEGFR-2 receptor. [Joikov et al., EMBO J, 16:(13):3898-3911 (1997).] It has also been demonstrated that a mutant VEGF-C, in which a single cysteine at position 156 is either substituted by another amino acid or deleted, loses the ability to bind VEGFR-2 but remains capable of binding and activating VEGFR-3 [International Patent Publication No. WO 98/33917]. In mouse embryos, VEGF-C mRNA is expressed primarily in the allantois, jugular area, and the metanephros. [Joukov et al., J Cell Physiol 173:211-215 (1997)]. VEGF-C is involved in the regulation of lymphatic angiogenesis: when VEGF-C was overexpressed in the skin of transgenic mice, a hyperplastic lymphatic vessel network was observed, suggesting that VEGF-C induces lymphatic growth [Jeltsch et al., Science, 276:1423-1425 (1997)]. Continued expression of VEGF-C in the adult also indicates a role in maintenance of differentiated lymphatic endothelium [Ferrara, J Mol Med 77:527-543 (1999)]. VEGF-C also shows angiogenic properties: it can stimulate migration of bovine capillary endothelial (BCE) cells in collagen and promote growth of human endothelial cells [see, e.g., International Patent Publication No. WO 98/33917, incorporated herein by reference]. VEGF-C₁₅₆S is a VEGF-C cysteine deletion variant that binds to VEGFR-3 but demonstrates reduced binding (relative to VEGF-C) to VEGFR-2. VEGF-C₁₅₆S and related ligands specific for VEGFR-3 that may be used in the present invention are described in U.S. Pat. No. 6,130,071, which specifically incorporated by reference in its entirety. VEGF-C materials and methods are described in U.S. Pat. Nos. 6,245530 and 6,221,839, incorporated herein by reference.

VEGF-D is structurally and functionally most closely related to VEGF-C [see International Patent Publ. No. WO 98/07832 and U.S. Pat. No. 6,235,713, incorporated herein by reference]. Like VEGF-C, VEGF-D is initially expressed as a prepro-peptide that undergoes N-terminal and C-terminal proteolytic processing, and forms non-covalently linked dimers. VEGF-D stimulates mitogenic responses in endothelial cells in vitro. During embryogenesis, VEGF-D is expressed in a complex temporal and spatial pattern, and its expression persists in the heart, lung, and skeletal muscles in adults. Isolation of a biologically active fragment of VEGF-D designated VEGF-DΔNΔC, is described in International Patent Publication No. WO 98/07832, incorporated herein by reference. VEGF-DΔNΔC consists of amino acid residues 93 to 201 of VEGF-D linked to the affinity tag peptide FLAG®.

VEGF-A was originally purified from several sources on the basis of its mitogenic activity toward endothelial cells, and also by its ability to induce microvascular permeability, hence it is also called vascular permeability factor (VPF). VEGF-A has subsequently been shown to induce a number of biological processes including the mobilization of intracellular calcium, the induction of plasminogen activator and plasminogen activator inhibitor-1 synthesis, promotion of monocyte migration in vitro, induction of antiapoptotic protein expression in human endothelial cells, induction of fenestrations in endothelial cells, promotion of cell adhesion molecule expression in endothelial cells and induction of nitric oxide mediated vasodilation and hypotension [Ferrara, J Mol Med 77: 527-543 (1999); Neufeld et al., FASEB J 13: 9-22 (1999); Zachary, Intl J Biochem Cell Bio 30: 1169-1174 (1998)].

VEGF-A is a secreted, disulfide-linked homodimeric glycoprotein composed of 23 kD subunits. Five human VEGF-A isoforms of 121, 145, 165, 189 or 206 amino acids in length (VEGF₁₂₁₋₂₀₆), encoded by distinct mRNA splice variants, have been described, all of which are capable of stimulating mitogenesis in endothelial cells. However, each isoform differs in biological activity, receptor specificity, and affinity for cell surface- and extracellular matrix-associated heparan-sulfate proteoglycans, which behave as low affinity receptors for VEGF-A. VEGF₁₂₁ does not bind to either heparin or heparan-sulfate; VEGF₁₄₅ and VEGF₁₆₅ (GenBank Acc. No. M32977) are both capable of binding to heparin; and VEGF₁₈₉ and VEGF₂₀₆ show the strongest affinity for heparin and heparan-sulfates. VEGF₁₂₁, VEGF₁₄₅, and VFGF₁₆₅ are secreted in a soluble form, although most of VEGF₁₆₅ is confined to cell surface and extracellular matrix proteoglycans, whereas VEGF₁₈₉ and VEGF₂₀₆ remain associated with extracellular matrix. Both VEGF₁₈₉ and VEGF₂₀₆ can be released by treatment with heparin or heparinase, indicating that these isoforms are bound to extracellular matrix via proteoglycans. Cell-bound VEGF₁₈₉ can also be cleaved by proteases such as plasmin, resulting in release of an active soluble VEGF₁₁₀. Most tissues that express VEGF are observed to express several VEGF isoforms simultaneously, although VEGF121 and VEGF₁₆₅ are the predominant forms, whereas VEGF₂₀₆ is rarely detected [Ferrara, J Mol Med 77:527-543 (1999)]. VEGF₁₄₅ differs in that it is primarily expressed in cells derived from reproductive organs [Neufeld et al., FASEB J 13:9-22 (1999)].

The pattern of VEGF-A expression suggests its involvement in the development and maintenance of the normal vascular system, and in angiogenesis associated with tumor growth and other pathological conditions such as rheumatoid arthritis. VEGF-A is expressed in embryonic tissues associated with the developing vascular system, and is secreted by numerous tumor cell lines. Analysis of mice in which VEGF-A was knocked out by targeted gene disruption indicate that VEGF-A is critical for survival, and that the development of the cardiovascular system is highly sensitive to VEGF-A concentration gradients. Mice lacking a single copy of VEGF-A die between day 11 and 12 of gestation. These embryos show impaired growth and several developmental abnormalities including defects in the developing cardiovasculature. VEGF-A is also required post-natally for growth, organ development, regulation of growth plate morphogenesis and endochondral bone formation. The requirement for VEGF-A decreases with age, especially after the fourth postnatal week. In mature animals, VEGF-A is required primarily for active angiogenesis in processes such as wound healing and the development of the corpus luteum. (Neufeld et al., FASEB J13:9-22 (1999); Ferrara, J Mol Med 77:527-543 (1999)]. VEGF-A expression is influenced primarily by hypoxia and a number of hormones and cytokines including epidermal growth factor (EGF), TGF-β, and various interleukins. Regulation occurs transcriptionally and also post-transcriptionally such as by increased mRNA stability [Ferrara, J Mol Med 77:527-543 (1999)].

Four additional members of the VEGF subfamily have been identified in poxviruses, which infect humans, sheep and goats. The orf virus-encoded VEGF-E and NZ2 VEGF are potent mitogens and permeability enhancing factors. Both show approximately 25% amino acid identity to mammalian VEGF-A, and are expressed as disulfide-liked homodimers. Infection by these viruses is characterized by pustular dermititis which may involve endothelial cell proliferation and vascular permeability induced by these viral VEGF proteins. [Ferrara, J Mol Med 77:527-543 (1999); Stacker and Achen, Growth Factors 17:1-11 (1999)]. VEGF-like proteins have also been identified from two additional strains of the orf virus, D1701 [GenBank Acc. No. AF106020; described in Meyer et al., EMBO J 18:363-374 (1999)] and NZ10 [described in International Patent Application PCT/US99/25869, incorporated herein by reference]. These viral VEGF-like proteins have been shown to bind VEGFR-2 present on host endothelium, and this binding is important for development of infection and viral induction of angiogenesis [Meyer et al., EMBO J 18:363-374 (1999); International Patent Application PCT/US99/25869].

### G. METHOD OF TREATING VEGFC RELATED DISORDERS

The present invention also involves, in another embodiment, the diagnosis and treatment of pathologies characterized by ligand-mediated activity of VEGFR-3. There are numerous disorders that may thus benefit from an intervention including but not limited to cancer, chronic inflammatory diseases, rheumatoid arthritis, psoriasis, diabetic retinopathy, and the like. In particular embodiments, the therapeutic methods of the invention are used in the treatment of lymphatic disorders. The cells of the invention may be isolated from a patient suspected of having such a disorder that is mediated through the binding of VEGFC to VEGFR-3.

By “lymphatic disorder” is meant any clinical condition affecting the lymphatic system, including but not limited to lymphedemas, lymphangiomas, lymphangiosarcomas lymphangiomatosis, lymphangiectasis, and cystic hygroma. Preferred embodiments are methods of screening a human subject for an increased risk of developing a lymphedema disorder, i.e., any disorder that physicians would diagnose as lymphedema and that is characterized by swelling associated with lymph accumulation, other than lymphedemas for which non-genetic causes (e.g., parasites, surgery) are known. By way of example, lymphedema disorders include Milroy-Nonne (OMIM 153100) syndrome-early onset lymphedema [Milroy, N.Y Med. J., 56:505-508 (1892); and Dale, J. Med. Genet., 22: 274-278 (1985)] and lymphedema praecox (Meige syndrome, OMIM 153200)-late onset lymphedema [Lewis et al., J. Ped., 104:641-648 (1984); Holmes et al., Pediatrics 61:575-579 (1978); and Wheeler et al., Plastic Reconstructive Surg., 67:362-364 (1981)] which generally are described as separate entities, both characterized by dominant inheritance. However, there is confusion in the literature about the separation of these disorders. In Milroy's syndrome, the presence of edema, which is usually more severe in the lower extremities, is seen from birth. Lymphedema praecox presents in a similar fashion but the onset of swelling is usually around puberty. Some cases have been reported to develop in the post-pubertal period. In the particular analyses described herein, the lymphedema families showing linkage to 5q34-q35 show an early onset for most affected individuals, but individuals in these pedigrees have presented during or after puberty.

Particularly contemplated for treatment according to the present invention are hereditary lymphedemas with an identifiable genetic cause. For example, International Patent Publication No. WO 005/58511 describes screening and therapy for lymphedemas involving VEGFR-3 mutations. The ability to isolate lymphatic endothelial cells from such patients permit improved protein-or gene based therapies by contacting target cells ex vivo with the therapeutic agent and reintroducing the cells.

In addition, the other types of disorders that may be treated, according to the present invention, such disorders are limited only by the involvement of VEGFC and/or VEGFR-3. Thus, it is contemplated that, for example, a wide variety of tumors may be assessed using the cells of the present invention including cancers of the brain (glioblastoma, astrocytoma, oligodendroglioma, ependymomas), lung, liver, spleen, kidney, lymph node, pancreas, small intestine, blood cells, colon, stomach, breast, endometrium, prostate, testicle, ovary, skin, head and neck, esophagus, bone marrow, blood or other tissue. Cells isolated from patients of having these diseases will be used to provide therapy to the individual.

In many contexts, in providing the therapy, it is not necessary that the tumor cell be killed or induced to undergo normal cell death or “apoptosis.” Rather, to accomplish a meaningful treatment, all that is required is that the tumor growth be slowed to some degree or localized to a specific area and inhibited from spread to disparate sites. It may be that the tumor growth is completely blocked, however, or that some tumor regression is achieved. Clinical terminology such as “remission” and “reduction of tumor” burden also are contemplated given their normal usage. In the context of the present invention, the therapeutic effect may result from an inhibition of angiogenesis and/or an inhibition of lymphangiogenesis.

I. Genetic Based Therapies

The cells isolated by the present invention may be treated using gene based therapy provided in the form of a nucleic acid, and reintroduced into the patient in order to effect ex vivo gene therapy. In an ex vivo embodiment, cells from the patient are removed and maintained outside the body for at least some period of time. During this period, a therapy is delivered, after which the cells are reintroduced into the patient; hopefully, any tumor cells in the sample have been killed. Specifically, the cells may be contacted with an expression construct capable of providing a therapeutic gene to the lymphatic or blood vascular cells of the tumor in a manner to allow the inhibition of VEGFR-3 in that vasculature.

For these embodiments, an exemplary expression construct comprises a virus or engineered construct derived from a viral genome. The expression construct generally comprises a nucleic acid encoding the therapeutic gene to be expressed and also additional regulatory regions that will effect the expression of the gene in the cell to which it is administered. Such regulatory regions include for example promoters, enhancers, polyadenylation signals and the like.

It is now widely recognized that DNA may be introduced into a cell using a variety of viral vectors. In such embodiments, expression constructs comprising viral vectors containing the genes of interest may be adenoviral (see for example, U.S. Pat. No. 5,824,544; U.S. Pat. No. 5,707,618; U.S. Pat. No. 5,693,509; U.S. Pat. No. 5,670,488; U.S. Pat. No. 5,585,362; each incorporated herein by reference), retroviral (see for example, U.S. Pat. No. 5,888,502; U.S. Pat. No. 5,830,725; U.S. Pat. No. 5,770,414; U.S. Pat. No. 5,686,278; U.S. Pat. No. 4,861,719 each incorporated herein by reference), adeno-associated viral (see for example, U.S. Pat. No. 5,474,935; U.S. Pat. No. 5,139,941; U.S. Pat. No. 5,622,856; U.S. Pat. No. 5,658,776; U.S. Pat. No. 5,773,289; U.S. Pat. No. 5,789,390; U.S. Pat. No. 5,834,441; U.S. Pat. No. 5,863,541; U.S. Pat. No. 5,851,521; U.S. Pat. No. 5,252,479 each incorporated herein by reference), an adenoviral-adenoassociated viral hybrid (see for example, U.S. Pat. No. 5,856,152 incorporated herein by reference) or a vaccinia viral or a herpesviral (see for example, U.S. Pat. No. 5,879,934; U.S. Pat. No. 5,849,571; U.S. Pat. No. 5,830,727; U.S. Pat. No. 5,661,033; U.S. Pat. No. 5,328,688 each incorporated herein by reference) vector.

In other embodiments, non-viral delivery is contemplated. These include calcium phosphate precipitation (Graham and Van Der Be, Virology, 52:456467, 1973; Chen and Okayama, Mol. Cell Biol., 7:2745-2752, 1987; Rippe et al., Mol. Cell Biol., 10:689-695, 1990) DEAE-dextran (Gopal, Mol. Cell Biol., 5:1188-1190, 1985), electroporation (Tur-Kaspa et al., Mol. Cell Biol., 6:716-718, 1986; Potter et al., Proc. Nat. Acad. Sci. USA, 81:7161-7165, 1984), direct microinjection (Harland and Weintraub, J. Cell Biol., 101:1094-1099, 1985.), DNA-loaded liposomes (Nicolau and Sene, Biochem. Biophys. Acta, 721:185-190, 1982; Fraley et al., Proc. Natl. Acad. Sci. USA, 76:3348-3352, 1979; Felgner, Sci Am. 276(6):102-6, 1997; Felgner, Hum Gene Ther. 7(15):1791-3, 1996), cell sonication (Fechheimer et al., Proc. Natl. Acad. Sci. USA, 84:8463-8467, 1987), gene bombardment using high velocity microprojectiles (Yang er al., Proc. Natl. Acad. Sci USA, 87:9568-9572, 1990), and receptor-mediated transfection (Wu and Wu, J. Biol. Chem., 262:4429-4432, 1987; Wu and Wu, Biochemistry, 27:887-892, 1988; Wu and Wu, Adv. Drug Delivery Rev., 12:159-167, 1993).

In a particular embodiment of the invention, the expression construct (or indeed the peptides discussed above) may be entrapped in a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, In: Liver diseases, targeted diagnosis and therapy using specific receptors and ligands, Wu G, Wu C ed., New York: Marcel Dekker, pp. 87-104, 1991). The addition of DNA to cationic liposomes causes a topological transition from liposomes to optically birefringent liquid-crystalline condensed globules (Radler et al., Science, 275(5301):8104, 1997). These DNA-lipid complexes are potential non-viral vectors for use in gene therapy and delivery.

Liposome-mediated nucleic acid delivery and expression of foreign DNA in vitro has been very successful. Also contemplated in the present invention are various commercial approaches involving “lipofection” technology. In certain embodiments of the invention, the liposome may be complexed with a hemagglutinating virus (HVJ). This has been shown to facilitate fusion with the cell membrane and promote cell entry of liposome-encapsulated DNA (Kaneda et al., Science, 243:375-378, 1989). In other embodiments, the liposome may be complexed or employed in conjunction with nuclear nonhistone chromosomal proteins (HMG-1) (Kato et al., J. Biol. Chem., 266:3361-3364, 1991). In yet further embodiments, the liposome may be complexed or employed in conjunction with both HVJ and HMG-1. In that such expression constructs have been successfully employed in transfer and expression of nucleic acid in vitro and in vivo, then they are applicable for the present invention.

Other vector delivery systems that can be employed to deliver a nucleic acid encoding a therapeutic gene into cells include receptor-mediated delivery vehicles. These take advantage of the selective uptake of macromolecules by receptor-mediated endocytosis in almost all eukaryotic cells. Because of the cell type-specific distribution of various receptors, the delivery can be highly specific (Wu and Wu, 1993, supra).

Receptor-mediated gene targeting vehicles generally consist of two components: a cell receptor-specific ligand and a DNA-binding agent. Several ligands have been used for receptor-mediated gene transfer. The most extensively characterized ligands are asialoorosomucoid (ASOR) (Wu and Wu, 1987, supra) and transferrin (Wagner et al., Proc. Nat'l. Acad Sd. USA, 87(9):3410-3414, 1990). Recently, a synthetic neoglycoprotein, which recognizes the same receptor as ASOR, has been used as a gene delivery vehicle (Ferkol et al., FASEB J., 7:1081-1091, 1993; Perales et al., Proc. Nati. Acad. Sci., USA 91:4086-4090, 1994) and epidermal growth factor (EGF) has also been used to deliver genes to squamous carcinoma cells (Myers, EPO 0273085).

In other embodiments, the delivery vehicle may comprise a ligand and a liposome. For example, Nicolau et al. (Methods Enzymol., 149:157-176, 1987) employed lactosyl-ceramide, a galactose-terminal asialganglioside, incorporated into liposomes and observed an increase in the uptake of the insulin gene by hepatocytes. Thus, it is feasible that a nucleic acid encoding a therapeutic gene also may be specifically delivered into a particular cell type by any number of receptor-ligand systems with or without liposomes.

In another embodiment of the invention, the expression construct may simply consist of naked recombinant DNA or plasmids. Transfer of the construct may be performed by any of the methods mentioned above that physically or chemically permeabilize the cell membrane. This is applicable particularly for transfer in vitro, however, it may be applied for in vivo use as well. Dubensky et al. (Proc. Nat. Acad. Sci. USA, 81:7529-7533, 1984) successfully injected polyomavirus DNA in the form of CaPO₄ precipitates into liver and spleen of adult and newborn mice demonstrating active viral replication and acute infection. Benvenisty and Neshif (Proc. Nat. Acad. Sci. USA, 83:9551-9555, 1986) also demonstrated that direct intraperitoneal injection of CaPO₄ precipitated plasmids results in expression of the transfected genes.

Another embodiment of the invention for transferring a naked DNA expression construct into cells may involve particle bombardment. This method depends on the ability to accelerate DNA coated microprojectiles to a high velocity allowing them to pierce cell membranes and enter cells without killing them (Klein et al., Nature, 327:70-73, 1987). Several devices for accelerating small particles have been developed. One such device relies on a high voltage discharge to generate an electrical current, which in turn provides the motive force (Yang et al., Proc. Natl. Acad. Sci USA, 87:9568-9572, 1990). The microprojectiles used have consisted of biologically inert substances such as tungsten or gold beads.

Those of skill in the art are well aware of how to apply gene delivery to in vivo and ex vivo situations. For viral vectors, one generally will prepare a viral vector stock. Depending on the kind of virus and the titer attainable, one will deliver 1×10⁴, 1×10⁵, 1×10⁶, 1×10⁷, 1×10⁸, 1×10⁹, 1×10¹⁰, 1×10¹¹ or 1×10¹² infectious particles to the patient. Similar figures may be extrapolated for liposomal or other non-viral formulations by comparing relative uptake efficiencies. Formulation as a pharmaceutically acceptable composition is discussed below.

Various routes are contemplated for various tumor types. The section below on routes contains an extensive list of possible routes. For practically any tumor, systemic delivery is contemplated. This will prove especially important for attacking microscopic or metastatic cancer. Where discrete tumor mass may be identified, a variety of direct, local and regional approaches may be taken. For example, the tumor may be directly injected with the expression vector or protein. A tumor bed may be treated prior to, during or after resection. Following resection, one generally will deliver the vector by a catheter left in place following surgery.

H. Immunotherapies

Immunotherapeutics, generally, rely on the use of immune effector cells and molecules to target and destroy cancer cells. The immune effector may be, for example, an antibody specific for some marker on the surface of a tumor cell. The antibody alone may serve as an effector of therapy or it may recruit other cells to actually effect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells.

In the context of the present invention, it is possible that the antibody, antibodies, antibody conjugates or immune effector cells target the selected tumor for therapy and the peptides of the present invention that are combined with the immunotherapy target the vasculature of the tumor thereby having a combined therapeutic effect.

The general approach for combined therapy is discussed below. In the context of the present invention, seeing as it has been determined that 2E11D11 and anti-podoplanin antibodies are capable of specifically recognizing lymphatic endothelial cells, it will be possible to target these cells with cytotoxic agents. As such, lymphangiogenesis associated with various cancers may be inhibited using the methods described herein.

In some embodiments, the antibodies may be used to target therapeutic proteins to the lymphatic endothelial cells. These therapies will be particularly useful as anti-lymphangiogenesis and/or anti-angiogenic treatments, however it is contemplated that the instant invention is not limited to these beneficial effects. Administration of the compositions can be systemic or local and may comprise a single site injection of a therapeutically effective amount of the protein. Any route known to those of skill in the art for the administration of a therapeutic composition of the invention is contemplated including for example, intravenous, intramuscular, subcutaneous or a catheter for long term administration. Alternatively, it is contemplated that the therapeutic composition may be delivered to the patient at multiple sites. The multiple administrations may be rendered simultaneously or may be administered over a period of several hours. In certain cases it may be beneficial to provide a continuous flow of the therapeutic composition. Additional therapy may be administered on a period basis, for example, daily, weekly or monthly. In addition, chemotherapeutic agents may also be target to the lymphatic endothelial cells. Such agents that will be useful in the therapeutic applications of the present invention are discussed in further detail below.

III. Combined Therapy with Immunotherapy, Traditional Chemo- or Radiotherapy

Tumor cell resistance to DNA damaging agents represents a major problem in clinical oncology. One goal of current cancer research is to find ways to improve the efficacy of chemo- and radiotherapy. One way is by combining such traditional therapies with gene therapy. For example, the herpes simplex-thymidine kinase (HS-tk) gene, when delivered to brain tumors by a retroviral vector system, successfully induced susceptibility to the antiviral agent ganciclovir (Culver et al., 1992). One embodiment of the present invention, it is contemplated that the peptides of the present invention may be administered in conjunction with chemo- or radiotherapeutic intervention, immunotherapy, or with other anti-angiogenic/anti-lymphangiogenic therapy.

To kill cells, inhibit cell growth, inhibit metastasis, inhibit angiogenesis or otherwise reverse or reduce the malignant phenotype of tumor cells, using the methods and compositions of the present invention, one would generally contact a “target” cell, a tumor or its vasculature with the at least two different therapeutic compositions. These compositions would be provided in a combined amount effective to kill or inhibit proliferation of the cancer by killing and/or inhibiting the proliferation of the cancer cells and/or the endothelia of blood and lymphatic vessels supplying and serving the cancer cells. This process may involve contacting the cells with the peptide or expression construct and the agent(s) or factor(s) at the same time. This may be achieved by contacting the cell with a single composition or pharmacological formulation that includes both agents, or by contacting the cell with two distinct compositions or formulations, at the same time.

Alternatively, the two different treatments may be separated by intervals ranging from minutes to weeks. In embodiments where the two therapies are administered separately, one would generally ensure that a significant period of time did not expire between the time of each delivery, such that the first and second therapy would still be able to exert an advantageously combined effect. In such instances, it is contemplated that one would administer both modalities within about 12-24 hours of each other and, more preferably, within about 6-12 hours of each other, with a delay time of only about 12 hours being most preferred. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations. Repeated treatments with one or both therapies is specifically contemplated. In specific embodiments, an anti-cancer therapy may be delivered which directly attacks the cancer cells in a manner to kill, inhibit or necrotize the cancer cell, in addition a therapeutic composition based an antiangiogenic and/or anti-lymphangiogenic effect also is administered. The antilymphangiogenic compositions may be administered following the other anti-cancer agent, before the other anti-cancer agent or indeed at the same time as the other anti-cancer agent.

Agents or factors suitable for use in a combined therapy are any chemical compound or treatment method that induces DNA damage when applied to a cell. Such agents and factors include radiation and waves that induce DNA damage such as, γ-irradiation, X-rays, UV-irradiation, microwaves, electronic emissions, and the like. A variety of chemical compounds, also described as “chemotherapeutic agents,” function to induce DNA damage, all of which are intended to be of use in the combined treatment methods disclosed herein. Chemotherapeutic agents contemplated to be of use, include, e.g., adriamycin, 5-fluorouracil (5FU), etoposide (VP-16), camptothecin, actinomycin-D, mitomycin C, cisplatin (CDDP) and even hydrogen peroxide. The invention also encompasses the use of a combination of one or more DNA damaging agents, whether radiation-based or actual compounds, such as the use of X-rays with cisplatin or the use of cisplatin with etoposide.

In treating cancer according to the invention, one would contact the tumor cells and/or the endothelia of the tumor vessels with an agent in addition to the antilymphangiogenic therapeutic agent. This may be achieved by irradiating the localized tumor site with radiation such as X-rays, UV-light, γ-rays or even microwaves. Alternatively, the tumor cells may be contacted with the agent by administering to the subject a therapeutically effective amount of a pharmaceutical composition comprising a compound such as, adriamycin, 5-fluorouracil, etoposide, camptothecin, actinomycin-D, mitomycin C, or cisplatin. Kinase inhibitors also contemplated to be useful in combination therapies with the peptides of the present invention. The agent may be prepared and used as a combined therapeutic composition, or kit, by combining it with a VEGF-C/D inhibitor peptide such as those described in U.S. Patent Application No. 60/262,476, filed Jan. 17, 2001, incorporated herein by reference.

Agents that directly cross-link nucleic acids, specifically DNA, are envisaged to facilitate DNA damage leading to a synergistic, antineoplastic combination with antilymphangiogenic agents. Agents such as cisplatin, and other DNA alkylating agents may be used. Cisplatin has been widely used to treat cancer, with efficacious doses used in clinical applications of 20 mg/m² for 5 days every three weeks for a total of three courses. Cisplatin is not absorbed orally and must therefore be delivered via injection intravenously, subcutaneously, intratumorally or intraperitoneally.

Agents that damage DNA also include compounds that interfere with DNA replication, mitosis and chromosomal segregation. Such chemotherapeutic compounds include adriamycin, also known as doxorubicin, etoposide, verapamil, podophyllotoxin, and the like. Widely used in a clinical setting for the treatment of neoplasm, these compounds are administered through bolus injections intravenously at doses ranging from 25-75 mg/m² at 21 day intervals for adriamycin, to 35-50 mg/m² for etoposide intravenously or double the intravenous dose orally.

Agents that disrupt the synthesis and fidelity of nucleic acid precursors and subunits also lead to DNA damage. As such a number of nucleic acid precursors have been developed. Particularly useful are agents that have undergone extensive testing and are readily available. As such, agents such as 5-fluorouracil (5-FU), are preferentially used by neoplastic tissue, making this agent particularly useful for targeting to neoplastic cells. Although quite toxic, 5-FU, is applicable in a wide range of carriers, including topical, however intravenous administration with doses ranging from 3 to 15 mg/kg/day being commonly used.

By way of example the following is a list of chemotherapeutic agents and the cancers which have been shown to be managed by administration of such agents. Combinations of these chemotherapeutics with the peptides of the present invention may prove to be useful in amelioration of various neoplastic disorders. Examples of these compounds include adriamycin (also known as doxorubicin), VP-16 (also known as etoposide), and the like, daunorubicin (intercalates into DNA, blocks DNA-directed RNA polymerase and inhibits DNA synthesis); mitomycin (also known as mutamycin and/or mitomycin-C) is an antibiotic isolated from the broth of Streptomyces caespitosus which has been shown to have antitumor activity; Actinomycin D also may be a useful drug to employ in combination with the peptides of the present invention because tumors which fail to respond to systemic treatment sometimes respond to local perfusion with dactinomycin which also is known to potentiate radiotherapy. It also is used in combination with primary surgery, radiotherapy, and other drugs, particularly vincristine and cyclophosphamide and has been found to be effective against Ewing's tumor, Kaposi's sarcoma, and soft-tissue sarcomas, choriocarcinoma, metastatic testicular carcinomas, Hodgkin's disease and non-Hodgkin's lymphomas.

Bleomycin is a mixture of cytotoxic glycopeptide antibiotics isolated from a strain of Streptomyces verticillus, is effective in the management of the following neoplasms either as a single agent or in proven combinations with other approved chemotherapeutic agents in squamous cell carcinoma such as head and neck (including mouth, tongue, tonsil, nasopharynx, oropharynx, sinus, palate, lip, buccal mucosa, gingiva, epiglottis, larynx), skin, penis, cervix, and vulva. It has also been used in the treatment of lymphomas and testicular carcinoma.

Cisplatin has been widely used to treat cancers such as metastatic testicular or ovarian carcinoma, advanced bladder cancer, head or neck cancer, cervical cancer, lung cancer or other tumors and may be a useful combination with the peptides of the present invention. VP16 (etoposide) and is used primarily for treatment of testicular tumors, in combination with bleomycin and cisplatin, and in combination with cisplatin for small-cell carcinoma of the lung. It is also active against non-Hodgkin's lymphomas, acute nonlymphocytic leukemia, carcinoma of the breast, and Kaposi's sarcoma associated with acquired immunodeficiency syndrome (AIDS). Tumor Necrosis Factor [TNF; Cachectin] glycoprotein that kills some kinds of cancer cells, activates cytokine production, activates macrophages and endothelial cells, promotes the production of collagen and collagenases, is an inflammatory mediator and also a mediator of septic shock, and promotes catabolism, fever and sleep. TNF can be quite toxic when used alone in effective doses, so that the optimal regimens probably will use it in lower doses in combination with other drugs. Its immunosuppressive actions are potentiated by γ-interferon, so that the combination potentially is dangerous. A hybrid of TNF and interferon-α also has been found to possess anti-cancer activity.

Taxol an antimitotic agent original isolated from the bark of the ash tree, Taxus brevifolia, and its derivative paclitaxol have proven useful against breats cancer and may be used in the combination therapies of the present invention. Beneficial responses to vincristine have been reported in patients with a variety of other neoplasms, particularly Wilms' tumor, neuroblastoma, brain tumors, rhabdomyosarcoma, and carcinomas of the breast, bladder, and the male and female reproductive systems. Vinblastine also is indicated as a useful therapeutic in the same cancers as vincristine. The most frequent clinical use of vinblastine is with bleomycin and cisplatin in the curative therapy of metastatic testicular tumors. It is also active in Kaposi's sarcoma, neuroblastoma, and Letterer-Siwe disease (histiocytosis X), as well as in carcinoma of the breast and choriocarcinoma in women.

Melphalan also known as alkeran, L-phenylalanine mustard, phenylalanine mustard, L-PAM, or L-sarcolysin, is a phenylalanine derivative of nitrogen mustard. Melphalan is a bifunctional alkylating agent which is active against selective human neoplastic diseases. Melphalan is the active L-isomer of the D-isomer, known as medphalan, which is less active against certain animal tumors, and the dose needed to produce effects on chromosomes is larger than that required with the L-isomer. Melphalan is available in form suitable for oral administration and has been used to treat multiple myeloma. Available evidence suggests that about one third to one half of the patients with multiple myeloma show a favorable response to oral administration of the drug. Melphalan has been used in the treatment of epithelial ovarian carcinoma.

Cyclophosphamide is stable in the gastrointestinal tract, tolerated well and effective by the oral and parental routes and does not cause local vesication, necrosis, phlebitis or even pain. Chlorambucil, a bifunctional alkylating agent of the nitrogen mustard type that has been found active against selected human neoplastic diseases. Chlorambucil is indicated in the treatment of chronic lymphatic (lymphocytic) leukemia, malignant lymphomas including lymphosarcoma, giant follicular lymphoma and Hodgkin's disease. It is not curative in any of these disorders but may produce clinically useful palliation.

Other factors that cause DNA damage and have been used extensively include what are commonly known as γ-rays, X-rays, and/or the directed delivery of radioisotopes to tumor cells. Other forms of DNA damaging factors are also contemplated such as microwaves and UV-irradiation. It is most likely that all of these factors effect a broad range of damage DNA, on the precursors of DNA, the replication and repair of DNA, and the assembly and maintenance of chromosomes. Dosage ranges for X-rays range from daily doses of 50 to 200 roentgens for prolonged periods of time (3 to 4 weeks), to single doses of 2000 to 6000 roentgens. Dosage ranges for radioisotopes vary widely, and depend on the half-life of the isotope, the strength and type of radiation emitted, and the uptake by the neoplastic cells.

The skilled artisan is directed to “Remington's Pharmaceutical Sciences” 15th Edition, chapter 33, in particular pages 624-652. Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

The inventors propose that the regional delivery of the therapy to patients with VEGFR-3-linked cancers will be a very efficient method for delivering a therapeutically effective gene to counteract the clinical disease. Similarly, the chemo- or radiotherapy may be directed to a particular, aced region of the subjects body. Alternatively, systemic delivery of expression construct and/or the agent may be appropriate in certain circumstances, for example, where extensive metastasis has occurred.

In addition to the anticancer therapeutics discussed above, it is contemplated that the peptides of the invention may be combined with other angiogenesis inhibitors. The peptides of the present invention are expected to have both anti-lymphangiogenic and anti-angiogenic properties. Many anti-angiogenic drugs also may have anti-lymphangiogenic properties. http://cancertrals.nci.nih.gov/news/angio is a website maintained by the National Institutes of Health which provides current information on the trials presently being conducted with anti-angiogenic agents. These agents include, for example, Maristat (British Biotech, Annapolis Md.; indicated for non-small cell lung, small cell lung and breast cancers); AG3340 (Agouron, LaJolla, Calif.; for glioblastoma multiforme); COL-3 (Collagenex, Newtown Pa.; for brain tumors); Neovastat (Aeterna, Quebec, Canada; for kidney and non-small cell lung cancer) BMS-275291 (Bristol-Myers Squibb, Wallingford Conn.; for metastatic non-small cell ling cancer); Thalidomide (Celgen; for melanoma, head and neck cancer, ovarian, metastatic prostate, and Kaposi's sarcoma; recurrent or metastatic colorectal cancer (with adjuvants); gynecologic sarcomas, liver cancer; multiple myeloma; CLL, recurrent or progressive brain cancer, multiple myeloma, non-small cell lung, nonmetastatic prostate, refractory multiple myeloma, and renal cancer); Squalamine (Magainin Pharmaceuticals Plymouth Meeting, Pa.; non-small cell cancer and ovarian cancer); Endostatin (EntreMEd, Rockville, Md.; for solid tumors); SU5416 (Sugen, San Francisco, Calif.; recurrent head and neck, advanced solid tumors, stage EIB or IV breast cancer; recurrent or progressive brain (pediatric); Ovarian, AML; glioma, advanced malignancies, advanced colorectal, von-Hippel Lindau disease, advanced soft tissue; prostate cancer, colorectal cancer, metastatic melanoma, multiple myeloma, malignant mesothelioma: metastatic renal, advanced or recurrent head and neck, metastaic colorectal cancer); SU6668 (Sugen San Francisco, Calif.; advanced tumors); interferon-α; Anti-VEGF antibody (National Cancer Institute, Bethesda Md.; Genentech San Franscisco, Calif.; refractory solid tumors; metastatic renal cell cancer, in untreated advanced colorectal); EMD121974 (Merck KCgaA, Darmstadt, Germany; HIV related Kaposi's Sarcoma, progressive or recurrent Anaplastic Glioma ); Interleukin 12 (Genetics Institute, Cambridge, Mass.; Kaposi's sarcoma) and IM862 (Cytran, Kirkland, Wash.; ovarian cancer, untreated metastatic cancers of colon and rectal origin and Kaposi's sarcoma). The parenthetical information following the agents indicates the cancers against which the agents are being used in these trials. It is contemplated that any of these disorders may be treated with the peptides of the present invention either alone or in combination with the agents listed.

It is that the effects of any of these therapies on lymphatic eidothelial cells may now be tested using the lymphatic endothelial cells isolated by the present invention. The availability of methods of isolating these cells will allow the development of more effective treatment protocols for the management of disorders of lymphatic endothelial cells.

### F. ASSAY FORMATS FOR IDENTIFYING ADDITIONAL THERAPEUTIC AGENTS

The present invention also contemplates the use of the lymphatic endothelial cells of the present invention in the screening of compounds that modulate (increase or decrease) characteristics of these cells such as VEGFR-3 receptor activity, cell growth, lymphangiogenic potential and the like of these cells. These assays may make use of a variety of different formats and may depend on the kind of “activity” for which the screen is being conducted. Contemplated functional “read-outs” include VEGFR-3 binding to a substrate; ligand binding to a receptor, migration assays, or any other functional assay normally employed to monitor endothelial cell activity. Such functional assays for endothelial cells are well known to those of skill in the art and some exemplary assays have been described elsewhere in this document.

a. Assay Formats.

The present invention provides methods of screening for inhibitors of VEGFR-3 activity by monitoring such activity in the presence and absence of the candidate substance and comparing such results. It is contemplated that this screening technique will prove useful in the general identification of a compound that will serve the purpose of inhibiting, decreasing or preventing the VEGFR-3 activity. Such compounds will be useful in the treatment of various disorders, such as for example, lymphomas, lymphedema, solid cancers characterized by neovascularization and other disorders such as those discussed in PCT/US99/06133, specifically incorporated herein by reference as providing examples of disorders involving VEGFR-3 receptor and, specifically, disorders including but not limited to hereditary lymphedema, lymphedemas, lymphangiomas, lymphangiosarcomas, lymphangiomatosis, lymphangiectasis, and cystic hygroma.

In these embodiments, the present invention is directed to a method for determining the ability of a candidate substance to inhibit the VEGFR-3 activity of the lymphatic endothelial cells of the present invention. The method includes generally the steps of:
(i) providing an isolated lymphatic endothelial cell culture of the present invention;
(ii) contacting said culture with a candidate substance; and
(iii) comparing the activity or characteristics of the cell culture of step (iii) with the activity or characteristics of the cell culture observed in the absence of the candidate substance,
wherein an alteration in the activity or characteristics of the cell culture indicates that said candidate substance is a modulator of said cells.

To identify a candidate substance as being capable of modulating the activity or altering the characteristics of the cells of the present invention in the assay above, one would measure or determine the activity or characteristics in the absence of the added candidate substance. One would then add the candidate substance to the cell culture and determine the activity or characteristics in the presence of the candidate substance. A candidate substance which alters the activity relative to that observed in its absence is indicative of a candidate substance with modulatory capability.

While the above method generally describes activity or characteristics of the cells in a culture of the present invention. It should be understood that candidate substance may be an agent that alters the production of VEGFR-3, thereby increasing or decreasing the amount of VEGFR-3 present as opposed to the per unit activity of the VEGFR-3. Similarly, the candidate may be one which increases or decreases the growth of cells in number and/or size Moreover, while the above discussion is directed to using isolated lymphatic endothelial cell cultures, it should be understood that similar assays also may be set up to identify therapeutic agents that act on blood vascular endothelial cells or modulate receptors and components thereof.

b. Candidate Substances.

As used herein the term “candidate substance” refers to any molecule that is capable of modulating an activity or characteristic of lymphatic endothelial cells. In specific embodiments, the molecule is one which modulates VEGFR-3 binding activity with its ligand. Alternatively, the candidate substance may modulate a downstream effect of VEGFR-3 receptor/ligand interaction, e.g., receptor autophosphorylation. The candidate substance may be a protein or fragment thereof, a small molecule inhibitor, or even a nucleic acid molecule. It may prove to be the case that the most useful pharmacological compounds for identification through application of the screening assay will be compounds that are structurally related to other known modulators of VEGFR-3 activity. The active compounds may include fragments or parts of naturally-occurring compounds or may be only found as active combinations of known compounds which are otherwise inactive. However, prior to testing of such compounds in humans or animal models, it will be necessary to test a variety of candidates to determine which have potential as therapeutic agents.

Accordingly, the active compounds may include fragments or parts of naturally-occurring compounds or may be found as active combinations of known compounds which are otherwise inactive. Accordingly, the present invention provides screening assays to identify agents which modulate cellular VEGF receptors. It is proposed that compounds isolated from natural sources, such as animals, bacteria, fungi, plant sources, including leaves and bark, and marine samples may be assayed as candidates for the presence of potentially useful pharmaceutical agents.

It will be understood that the pharmaceutical agents to be screened could also be derived or synthesized from chemical compositions or man-made compounds. Thus, it is understood that the candidate substance identified by the present invention may be polypeptide, polynucleotide, small molecule inhibitors or any other inorganic or organic chemical compounds that may be designed through rational drug design starting from known modulators of VEGF receptors.

The candidate screening assays are simple to set up and perform. Thus, in assaying for a candidate substance, after obtaining an isolated lymphatic endothelial cell population of the present invention, one will admix a candidate substance with the cells of the population, under conditions which would allow a lymphatic endothelial cells specific measurable activity to occur or specific characteristic to be observed. In this fashion, one can measure the ability of the candidate substance to modulate the activity or characteristic of the cell in the absence of the candidate substance.

“Effective amounts” in certain circumstances are those amounts effective to reproducibly alter a given event, activity or phenotype from the cell in comparison to their normal levels. Compounds that achieve significant appropriate changes in activity will be used.

Significant changes in activity or functional characteristic, e.g., as measured using migration assays, cell proliferation assays, receptor binding, autophosphorylation and the like are represented by an increase/decrease in activity of at least about 30%-40% and most preferably, by changes of at least about 50%, with higher values of course being possible. The active compounds of the present invention also may be used for the generation of antibodies which may then be used in analytical and prepatory techniques for detecting and quantifying further such modulators.

The isolated cell cultures of the invention are amendable to numerous high throughput screening (HTS) assays known in the art. For a review see Jayawickreme and Kost, Curr. Opin. Biotechnol. 8: 629-634 (1997). Automated and miniaturized HTS assays are also contemplated as described for example in Houston and Banks Curr. Opin. Biotechnol. 8: 734-740 (1997)

There are a number of different libraries used for the identification of small molecule modulators including chemical libraries, natural product libraries and combinatorial libraries comprised or random or designed peptides, oligonucleotides or organic molecules. Chemical libraries consist of structural analogs of known compounds or compounds that are identified as hits or leads via natural product screening or from screeing against a potential therapeutic target. Natural product libraries are collections of products from microorganisms, animals, plants, insects or marine organisms which are used to create mixtures of screening by, e.g., fermentation and extractions of broths from soil, plant or marine organisms. Natural product libraries include polypeptides, non-ribosomal peptides and non-naturally occurring variants thereof. For a review see Science 282:63-68 (1998). Combinatorial libraries are composed of large numbers of peptides oligonucleotides or organic compounds as a mixture. They are relatively simple to prepare by traditional automated synthesis methods, PCR cloning or other synthetic methods. Of particular interest will be libraries that include peptide, protein, peptidomimetic, multiparallel synthetic collection, recombinatorial and polypeptide libraries. A review of combinatorial libraries and libraries created therefrom, see Myers Curr. Opin. Biotechnol. 8: 701-707 (1997). A candidate modulator identified by the use of various libraries described may then be optimized to modulate activity of the cells through, for example, rational drug design.

It will of course, be understood that all the screening methods of the present invention are useful in themselves notwithstanding the fact that effective candidates may not be found. The invention provides methods for screening for such candidates, not solely methods of finding them.

c. In vitro Assays.

In one particular embodiment, the invention encompasses various binding assays. These can include screening for inhibitors of ligand-receptor complexes or for molecules capable of binding to VEGFR-3, as a substitute of the receptor function and thereby altering the binding of the natural ligand to this receptor and affecting its activity. In such assays, the cells may be either free in solution, or fixed to a support. Either the ligand or the receptor on the cell may be labeled, thereby permitting determination of binding.

Such assays are highly amenable to automation and high throughput. High throughput screening of compounds is described in WO 84/03564. Large numbers of small peptide test compounds are synthesized on a solid substrate, such as plastic pins or some other surface. The peptide test compounds are reacted with the cells and washed Bound polypeptide is detected by various methods. Combinatorial methods for generating suitable peptide test compounds are specifically contemplated.

Of particular interest in this format will be the screening of a variety of different mutants of the natural ligand for the VEGFR-3 receptor on these cells. These mutants, including deletion, truncation, insertion and substitution mutants, will help identify which domains are involved with the ligand/receptor interaction. Once this region has been determined, it will be possible to identify which of these mutants, which have altered structure but retain some or all of the functions of this interaction.

Purified ligand can be coated directly onto plates for use in the aforementioned drug screening techniques. However, non-neutralizing antibodies to the polypeptide can be used to immobilize the polypeptide to a solid phase. Also, fusion proteins containing a reactive region (preferably a terminal region) may be used to link the ligand active region to a solid phase.

Other forms of in vitro assays include those in which functional readouts are taken. In such assays, the substance would be formulated appropriately, given its biochemical nature, and contacted with the cell. Depending on the assay, culture may be required The cell may then be examined by virtue of a number of different physiologic assays, as discussed above. Alternatively, molecular analysis may be performed in which the cells characteristics are examined. This may involve assays such as those for protein expression, enzyme function, substrate utilization, mRNA expression (including differential display of whole cell or polyA RNA) and others.

G. Use of Cells in Diagnostic Assays

In certain embodiments, the methods of the present invention may be used for the diagnosis of conditions or diseases with which the aberrations in the function or activity of components of the cells, e.g., VEGFR-3/ligand interaction. For example, the cells from a patient suspected of having a disorder associated with lymphatic endothelial cell may be isolated using the methods of the present invention. Polynucleotide sequences from the cells may be used in hybridization or PCR assays to detect the presence of disease related expression. Such methods may be qualitative or quantitative in nature and may include Southern or northern analysis, dot blot or other membrane-based technologies; PCR technologies; dip stick, pin, chip and ELISA technologies. All of these techniques are well known in the art and are the basis of many commercially available diagnostic kits.

In addition such assays may useful in evaluating the efficacy of a particular therapeutic treatment regime in animal studies, in clinical trials, or in monitoring the treatment of an individual patient in order to provide a basis for the diagnosis of disease, a normal or standard profile for e.g., VEGFR-3 receptor expression needs to be established. This generally involves obtaining lymphatic endothelial cells from normal subjects, and performing suitable hybridization or amplification of disease markers therefrom. Standard hybridization may be quantified by comparing the values obtained for normal subjects with a dilution series of the marker. Standard values obtained from normal samples may be compared with values obtained from cells samples from subjects being diagnosed for a given disorder. Deviation between standard and subject values establishes the presence of disease.

Once disease is established, a therapeutic agent is administered; and a treatment profile is generated. Such assays may be repeated on a regular basis to evaluate whether the values in the profile progress toward or return to the normal or standard pattern Successive treatment profiles may be used to show the efficacy of treatment over a period of several days or several months.

PCR as described in U.S. Pat. Nos. 4,683,195 and 4,965,188. Oligomers for use in such assays are generally chemically synthesized, but they maybe generated enzymatically or produced from a recombinant source as described herein above. Oligomers generally comprise two nucleotide sequences, one with sense orientation and one with antisense, employed under optimized conditions for identification of a specific gene or condition. The same two oligomers, nested sets of oligomers, or even a degenerate pool of oligomers may be employed under less stringent conditions for detection and/or quantitation of closely related DNA or RNA sequences.

Additionally, methods to quantitate the expression of a particular molecule include radiolabeling (Melby et al., J Immunol Methods 159: 235-44, 1993) or biotinylating (Duplaa et al., Anal Biochem 229-36, 1993) nucleotides, coamplification of a control nucleic acid, and standard curves onto which the experimental results are interpolated. Quantitation of multiple samples may be speeded up by running the assay in an ELISA format where the oligomer of interest is presented in various dilutions and a spectrophotometric or colorimetric response gives rapid quantitation. A definitive diagnosis of this type may allow health professionals to begin aggressive treatment and prevent further worsening of the condition. Similarly, further assays can be used to monitor the progress of a patient during treatment

### H. KITS

The present invention concerns kits for isolating lymphatic endothelial cells using the methods described above. Such kits may include kits, standard VEGF receptor ligands, buffers and the like. As the present invention identifies specific antibodies that may be employed to specifically detect lymphatic endothelial cells, either or both of such components may be provided in the kit The kits may thus comprise, in suitable container means, a lymphatic or blood vascular endothelial cell component to act as a standard, a first antibody that preferentially binds to lymphatic endothelial cells, and an immunodetection reagent.

Still other compositions of the present invention that can be supplied in a kit format are the lymphatic endothelial cells substantially free of other contaminating cells that are non-lymphatic in lineage and the blood vascular endothelial cells substantially free of other contaminating cells that are non-vascular in lineage. The cells may be supplied as a proliferating culture in a culture flask or may be provided as cryopreserved cells. The cell-based kits also may comprise suitable media, growth supplements and instructions for growth conditions to be used for growing the cells.

In certain embodiments, the first antibody that binds to the lymphatic endothelial cells may be bound to a solid support, such as a column matrix or well of a microtiter plate.

The immunodetection reagents of the kit may take any one of a variety of forms, including those detectable labels that are associated with or linked to the given antibody or antigen, and detectable labels that are associated with or attached to a secondary binding ligand. Exemplary secondary ligands are those secondary antibodies that have binding affinity for the first antibody or antigen, and secondary antibodies that have binding affinity for a human antibody.

Further suitable immunodetection reagents for use in the present kits include the two-component reagent that comprises a secondary antibody that has binding affinity for the first antibody or antigen, along with a third antibody that has binding affinity for the second antibody, the third antibody being linked to a detectable label.

The kits may further comprise a suitably aliquoted amounts of proliferating or cryopreserved cells, whether labeled or unlabeled, as may be used to prepare a standard curve for a detection assay.

The kits may contain antibody-label conjugates either in fully conjugated form, in the form of intermediates, or as separate moieties to be conjugated by the user of the kit The components of the kits may be packaged either in aqueous media or in lyophilized form.

The container means of the kits will generally include at least one vial, test tube, flask, bottle, syringe or other container means, into which the antibody or antigen may be placed, and preferably, suitably aliquoted Where a second or third binding ligand or additional component is provided, the kit will also generally contain a second, third or other additional container into which this ligand or component may be placed. The kits of the present invention will also typically include a means for containing the antibody, antigen, and any other reagent containers in close confinement for commercial sale. Such containers may include injection or blow-molded plastic containers into which the desired vials are retained.

### I. IMAGING LYMPHATIC ENDOTHELIAL CELLS

An additional use for methods of the present invention is in tissue imaging to determine the presence of lymphatic endothelial cells in particular tissue. The use of such diagnostic imaging is particularly suitable in obtaining an image of, for example, a tissue from a patient suffering from a lymphatic disorder. Additionally, lymphatic vessels in or near a tumor mass also may be imaged by the present invention. Previously, those of skill in the art have employed VEGFR-3 antibodies for imagining purposes as described for example in U.S. Pat. No. 6,107,046 (incorporated herein by reference). It is contemplated that the 2E11D11 and related antibodies described in the present invention may be employed for imaging in a manner analogous to the antibody-based methods disclosed in U.S. Pat. No. 6,107,046.

The imaging agents of the present invention (i.e., the antibodies or antibody derivatives described herein throughout) may be coupled either covalently or noncovalently to a suitable supramagnetic, paramagnetic, electron-dense, echogenic or radioactive agent to produce a targeted imaging agent. In such embodiments, the imaging agent will localize to the lymphatic endothelial cells and the area of localization be imaged using the above referenced techniques.

Many appropriate imaging agents are known in the art, as are methods of attaching the labeling agents to the peptides of the invention (see, e.g., U.S. Pat. No. 4,965,392, U.S. Pat. No. 4,472,509, U.S. Pat. No. 5,021,236 and U.S. Pat. No. 5,037,630, incorporated herein by reference). The imaging agents are administered to a subject in a pharmaceutically acceptable carrier, and allowed to accumulate at a target site having the lymphatic endothelial cells. This imaging agent then serves as a contrast reagent for X-ray, magnetic resonance, sonographic or scintigraphic imaging of the target site. The antibodies of the present invention are a convenient and important addition to the available arsenal of medical imaging tools for the diagnostic investigation of cancer, lymphedema and other lymphatic endothelial cell disorders. Of course, it should be understood that the imaging may be performed in vitro where tissue from the subject is obtained through a biopsy, and the presence of lymphatic endothelial cells is determined with the aid of the imaging agents described herein in combination with histochemical techniques for preparing and fixing tissues.

Paramagnetic ions useful in the imaging agents of the present invention include for example chromium (III), manganese (II), iron (III), iron (II), cobalt (II), nickel (II) copper (II), neodymium (III), samarium (III), ytterbium(I), gadolinium (III), vanadium (II), terbium (III), dysprosium (III), holmium (III) and erbium (III). Ions useful for X-ray imaging include but are not limited to lanthanum (III), gold(III), lead (II) and particularly bismuth (III). Radioisotopes for diagnostic applications include for example, ²¹¹astatine, ¹⁴carbon, ⁵¹chromium, ³⁶chlorine, ⁵⁷cobalt, ⁶⁷copper, ¹⁵²Eu, 67gallium, ³hydrogen, ¹²³iodine, ¹²⁵ iodine, ¹¹¹indium, ⁵⁹iron, ³²phosphorus, ¹⁸⁶rhenium, ⁷⁵selenium, ³⁵sulphur, ^{99m}technicium and ⁹⁰yttrium.

The antibodies of the present invention may be labeled according to techniques well known to those of skill in the are. For example, the peptides can be iodinated by contacting the peptide with sodium or potassium iodide and a chemical oxidizing agent such as sodium hypochlorite or an enzymatic oxidant such as lacteroxidase. Antibodies may be labeled with technicium-99m by ligand exchange, for example, by reducing pertechnate with stannous solution, chelating the reduced technicium onto a Sephadex column and applying the antibody to the column. These and other techniques for labeling proteins and peptides are well known to those of skill in the art.

### J. PHARMACEUTICAL COMPOSITIONS

In many aspects, the cells or other compositions discussed in the present invention will be used for clinical purposes. As such, it will be necessary to prepare these formulations as pharmaceutical compositions, i.e., in a form appropriate for in vivo applications. Generally, this will entail preparing compositions that are essentially free of pyrogens, as well as other impurities that could be harmful to humans or animals.

One will generally desire to employ appropriate salts and buffers to render delivery vectors stable and allow for uptake by target cells. Buffers also will be employed when recombinant cells are introduced into a patient. Aqueous compositions of the present invention comprise an effective amount of the peptide or an expression vector to cells, dissolved or dispersed in a pharmaceutically acceptable carrier or aqueous medium. Such compositions also are referred to as inocula. The phrase “pharmaceutically or pharmacologically acceptable” refer to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the vectors or cells of the present invention, its use in therapeutic compositions is contemplated. Supplementary active ingredients also can be incorporated into the compositions.

The active compositions of the present invention include classic pharmaceutical preparations. Administration of these compositions according to the present invention will be via any common route so long as the target tissue is available via that route. The pharmaceutical compositions may be introduced into the subject by any conventional method, e.g., by intravenous, intradermal, intramusclar, intramammary, intraperitoneal, intrathecal, retrobulbar, intrapulmonary (e.g., term release); by oral, sublingual, nasal, anal, vaginal, or transdermal delivery, or by surgical implantation at a particular site. The treatment may consist of a single dose or a plurality of doses over a period of time.

The active compounds may be prepared for administration as solutions of free base or pharmacologically acceptable salts in water suitably mixed with a surfactant, such as hydroxypropylcellulose. Dispersions also can be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial an antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle that contains the basic dispersion medium and the required other ingredients from those enumerated above. In the case of sterile powders for the preparation of sterile injectable solutions, the preferred methods of preparation are vacuuming and freeze drying techniques that yield a powder of the active ingredient plus any additional desired ingredient from a previously sterile-filtered solution thereof.

As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients also can be incorporated into the compositions.

For oral administration the compositions may be incorporated with excipients and used in the form of non-ingestible mouthwashes and dentifrices. A mouthwash may be prepared incorporating the active ingredient in the required amount in an appropriate solvent, such as a sodium borate solution (Dobell's Solution). Alternatively, the active ingredient may be incorporated into an antiseptic wash containing sodium borate, glycerin and potassium bicarbonate. The active ingredient may also be dispersed in dentifrices, including: gels, pastes, powders and slurries. The active ingredient may be added in a therapeutically effective amount to a paste dentifrice that may include water, binders, abrasives, flavoring agents, foaming agents, and humectants.

The compositions of the present invention may be formulated in a neutral or salt form. Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups also can be derived from inorganic bases such as, for example, sodium, potassium, ammonium, calcium, or ferric hydroxides, and such organic bases as isopropylamine, trimethylamine, histidine, procaine and the like.

The compositions of the present invention may be formulated in a neutral or salt form. Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups also can be derived from inorganic bases such as, for example, sodium, potassium, ammonium, calcium, or ferric hydroxides, and such organic bases as isopropylamine, trimethylamine, histidine, procaine and the like.

Upon formulation, solutions will be administered in a manner compatible with the dosage formulation and in such amount as is therapeutically effective. The formulations are easily administered in a variety of dosage forms such as injectable solutions, drug release capsules and the like. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration.

“Unit dose” is defined as a discrete amount of a therapeutic composition dispersed in a suitable carrier. For example, where polypeptides are being administered parenterally, the polypeptide compositions are generally injected in doses ranging from 1 μg/kg to 100 mg/kg body weight/day, preferably at doses ranging from 0.1 mg/kg to about 50 mg/kg body weight/day. Parenteral administration may be carried out with an initial bolus followed by continuous infusion to maintain therapeutic circulating levels of drug product. Those of ordinary skill in the art will readily optimize effective dosages and administration regimens as determined by good medical practice and the clinical condition of the individual patient.

The frequency of dosing will depend on the pharmacokinetic parameters of the agents and the routes of administration. The optimal pharmaceutical formulation will be determined by one of skill in the art depending on the route of administration and the desired dosage. See for example Remington's Pharmaceutical Sciences, 18th Ed. (1990, Mack Publ. Co, Easton Pa. 18042) pp 1435-1712, incorporated herein by reference. Such formulations may influence the physical state, stability, rate of in vivo release and rate of in vivo clearance of the administered agents. Depending on the route of administration, a suitable dose may be calculated according to body weight, body surface areas or organ size. Further refinement of the calculations necessary to determine the appropriate treatment dose is routinely made by those of ordinary skill in the art without undue experimentation, especially in light of the dosage information and assays disclosed herein as well as the pharmacokinetic data observed in animals or human clinical trials.

Appropriate dosages may be ascertained through the use of established assays for determining blood clotting levels in conjunction with relevant dose-response data. The final dosage regimen will be determined by the attending physician, considering factors that modify the action of drugs, e.g., the drug's specific activity, severity of the damage and the responsiveness of the patient, the age, condition, body weight, sex and diet of the patient, the severity of any infection, time of administration and other clinical factors. As studies are conducted, further information will emerge regarding appropriate dosage levels and duration of treatment for specific diseases and conditions.

In gene therapy embodiments employing viral delivery, the unit dose may be calculated in terms of the dose of viral particles being administered. Viral doses include a particular number of virus particles or plaque forming units (pfu). For embodiments involving adenovirus, particular unit doses include 10³, 10⁴, 10⁵, 10⁶, 10⁷, 10⁸, 10⁹, 10¹⁰, 10¹¹, 10¹², 10¹³ or 10¹⁴ pfu. Particle doses may be somewhat higher (10 to 100-fold) due to the presence of infection defective particles.

It will be appreciated that the pharmaceutical compositions and treatment methods of the invention may be useful in fields of human medicine and veterinary medicine. Thus the subject to be treated may be a mammal, preferably human or other animal. For veterinary purposes, subjects include for example, fire animals including cows, sheep, pigs, horses and goats, companion animals such as dogs and cats, exotic and/or zoo animals, laboratory animals including mice rats, rabbits, guinea pigs and hamsters; and poultry such as chickens, turkey ducks and geese.

### K. EXAMPLE

The following example presents preferred embodiments and techniques, but is not intended to be limiting. Those of skill in the art will, in light of the present disclosure, appreciate that many changes can be made in the specific materials and methods which are disclosed and still obtain a like or similar result without departing from the spirit and scope of the invention.

### Example 1

### Materials and Methods

The present example provides details of materials and methods employed throughout the application and in the Examples presented herein below.

Antibodies and growth factors. The primary antibodies used in immunofluorescence were mouse mAbs against human CD31 (Dako), vWF (Dako) or VEGFR-3 (clones 9D9F9, 2E11D11 and 7B3F9; Jussila et at., Cancer Res. 58:1599-1604, 1998), rabbit antiserum against human LYVE-1 (Banerji et al., J. Biol. Chem., 144(4)789-801, 1999), affinity purified rabbit anti-human podoplanin (Breiteneder-Geleff et al., et al., Am. J. Path., 154(2) 385-394, 1999) or rabbit anti-human VEGF-C (882; Joukov et al., EMBO J., 15:290-298, 1996). Monoclonal antibody against proliferating cell nuclear antigen (clone PC10) was from Santa Cruz Biotechnology. FITC- or TRITC-conjugated goat anti-rabbit IgG, goat anti-mouse IgG and donkey anti-mouse IgG were obtained from Jackson Immunoresearch. The rabbit antiserum against human VEGFR-2 was a kind gift from Lena Claesson-Welsh (Uppsala, Sweden) and affinity purified goat anti-human VEGFR-1 was from R&D Systems. Rabbit polyclonal antibodies against Akt, MAPK or CREB were from New England Biolabs. Basic FGF, recombinant human VEGF165 and recombinant mature human VEGF-D (consisting of residues Phe93 to Ser201) were from R&D. Recombinant human PIGF-1 was a kind gift from Graziella Persico (Naples, Italy). The recombinant human VEGF-C (Thr103 to Leu215), VEGF-C156S (Thr103 to Ile225), ORFV2-VEGF and human VEGFR-3-Ig were produced and purified as described earlier (Joukov et at., EMBO J., 16:3898-3911, 1997; Mäkinen et at., Nature Med.,7:199-205, 2001; Wise et al., Proc. Nat'l Acad Sci., 96:3071-3076, 1999). Wortmannin, LY294002, PD98059 and Bisindolylmaleimide I (GF109203X) were from Calbiochem and U0126 from Promega (Madison, Wisc.).

Cell culture. HMVE and HUVE cells were obtained from PromoCell (Heidelberg, Germany), cultured in endothelial cell medium provided by the supplier and used at passages 3 to 7. The murine Ba/F3 pre-B lymphocytes were cultured in DMEM supplemented with 10% fetal calf serum, glutamine and 2 ng/ml IL-3 (Calbiochem).

Immunofluorescence staining. Cells on glass coverslips were fixed in 4% paraformaldehyde (PFA) or methanol:acetone (1:1) for 10 min. If required, the cells were permeabilised with 0.1% TritonX-100 in PBS for 5 min. After blocking in 5% goat serum, the cells were shine with the primary antibodies for 30 min at room temperature, followed by incubation with FITC- or TRITC-conjugated secondary antibodies (15 μg/ml) for 30 min. Hoechst 33258 fluorochrome (Sigma, 0.5 μg/ml in PBS) was used for the staining of the nuclei. If cells were stained alive, the procedure was carried out on ice, followed by fixation in PFA.

Isolation of lymphatic and blood vascular endothelial cells. Monoclonal VEGFR-3 antibodies (clone 2E11D11) or polyclonal podoplanin antibodies, MACS colloidal super-paramagnetic MicroBeads conjugated to rat anti-mouse IgG1 or to goat anti-rabbit IgG antibodies (Miltenyi Biotech, Bergisch Gladbach, Germany), MACS MS separation columns and MiniMACS separator (Miltenyi Biotech) were used for cell sorting according to the instructions of the manufacturer.

Bioassay for VEGFR stimulation. Viability assays using Ba/F3 pre-B cells expressing VEGFR-2/EpoR (Achen et al., Proc Natl Acad Sci USA 95:548-53 1998; Stacker et al., J. Biol. Chem., 274:34884-34892, 1999) or VEGFR-3/EpoR (Achen et al., Eur. J. Biochem., 267: 2505-2515, 2000) were carried out as described earlier (Mäkinen et al., Nature Med.,7:199-205, 2001). For the generation of Ba/F3 VEGFR-1/EpoR cells, the chimeric receptor was constructed by introducing a BglII site into the human VEGFR-1 cDNA prior to the sequence encoding the transmembrane domain followed by ligation of BglII-NotI fragment consisting of the transmembrane and intracellular domains of mouse erythropoietin receptor (Achen et al., Eur. J. Biochem, 267: 2505-2515, 2000). The VEGFR-1/EpoR cDNA was subcloned into the pEF-BOS expression vector (Mizushima and Nagata, Nucleic Acid Res., 18:5322, 1990) and co-transfected into Ba/F3 cells with pCDNA3.1(+)Zeo vector (Invitrogen). Stable cell pools were generated by selection with 250 mg/ml zeocin.

Biosensor Analysis. All protein preparations were analysed for homogeneity and buffer exchanged by micropreparative size exclusion HPLC using a Superose 12 (3.2/30) column installed in a SMART™ system (Amersham Pharmacia Biotech, Uppsala, Sweden) immediately prior to use (Nice and Catimel, Bioessays, 21:339-352, 1999). The concentrations of VEGF-C and VFGF-D were determined by absorbance at 280 mn using E280 1% 1 cm of 0-65. Receptor domains were coupled to the carboxymethylated dextran layer of a CM5 sensor chip using standard amine coupling chemistry (Nice and Catimel, Bioessays, 21:339-352, 1999) for analysis of ligand binding using a BIAcore 2000 optical biosensor (BLAcore, Uppsala, Sweden). The levels immobilized were 3,000 RU and 7,000 RU for VEGFR-2 and VEGFR-3, respectively. Following immobilization, residual activated ester groups were blocked by treatment with 1 M ethanolamine hydrochloride pH 8.5 followed by washing with 10 mM diethylamine to remove non-covalently bound material. 10 mM diethylamine or 10 mM HCl was used to regenerate the sensor surface between analyses for VEGF-D or VEGF-C binding, respectively. Samples were diluted in running buffer (10 mM HEPES pH 7.4, 150 mM NaCl, 3.4 mM EDTA, 0.005% Tween 20). The apparent binding affinities of VEGF-C and VEGF-C156S to receptor domains were determined by analysis of the initial dissociation phase to obtain the kd, which was then used to constrain a global analysis of the association region of the curves, assuming a 1:1 Langmuirian model. Data were analysed using BIAevaluation 3.0 (BIAcore, Uppsala, Sweden) as described previously (Catimel et al., J. Chromatogr., 776:15-30, 1997).

Analysis of endothelial cell apoptosis. For the apoptosis assay, 70,000 cells per well were seeded into 24-well plates. Treatments were done in duplicates and apoptosis was detected by measuring cytoplasmic histone-associated DNA fragments using the death detection ELISA PLUS kit (Roche, Indianapolis, Ind.). The following ranges of growth factor concentrations were tested: bFGF 10-20 ng/ml, PIGF-1 50-1000 ng/ml, VEGF 10-50 ng/ml, VEGFC 50-1000 ng/ml, VEGF-D 50-1000 ng/ml, VEGF-C156S 50-1000 ng/ml and VEGF-E 50-1000 ng/ml. Annexin-V-FLUOS (Roche, Indianapolis, Ind.) was used to detect phosphatidylserine on the apoptotic cells by fluorescence microscopy according to the instructions of the manu The simultaneous staining with propidium iodide (1 μg/ml) was used for discriminating possible necrotic cells.

Western blot analysis. Endothelial cells were cultured on 35 mm dishes to near confluence, starved for 24 h in serum free medium and stimulated as indicated. Wortmannin (30 nM), LY294002 (10-20 μM), PD98059 (10-25 μM) or GF109203X (2.5-5 μM) were added 1-3 h before stimulation, where indicated. DMSO, into which the inhibitors were dissolved, was used as a control. After the stimulation, the cells were lysed in lysis buffer (50 mM HEPES, pH 7.5, 150 mM NaCl, 10 mM EDTA, 100 mM NaF, 1% TritonX-100 supplemented with 2 mM Na3VO4, 0.5 mM PMSF, 100 U/ml approtinin and 10 μg/ml leupeptin). Clarified lysates were separated by SDS-PAGE, transferred to nitrocellulose and immunoblotted using the phosphospecific antibodies for Akt-Ser473, Akt-Thr308, p42/44 MAPK-Thr202/Tyr204 or CREB-Ser133. The bound antibodies were detected using horseradish peroxidase conjugated secondary antibodies and enhanced chemiluminescence detection system. The blots were stripped and reprobed with antibodies against Akt, MAPK or CREB for quantification by reading the optical densities of the signals with Multi-Analyst 2.0.1 program (Bio-Rad).

Cell migration assay. Migration assays were performed in a 48-well chemotaxis Boyden chamber (Neuroprobe Inc.). Eight micron Nucleopore polycarbonate filters (Corning) were coated with 100 μg/ml of collagen type I (Upstate Biotechnology) overnight at +4° C. and air dried. The filters were placed over the lower chamber wells containing the growth factors in serum-free growth medium supplemented with 0.2% BSA. For blocking experiments, VEGF and VEGF-C156S were preincubated with a ten-fold molar excess of soluble human VEGFR-3 for 30 min. HMVE cells were suspended in the growth medium and 10,000 cells in 50 μl were added to each well in the upper chamber. The cells were allowed to migrate for 6 h at 37° C. after which the filter was fixed with cold methanol and stained with hematoxylin (Meyer). Non-migrated cells on the upper surface of the filter were removed by scraping with a cotton swab and the number of migrated cells was counted. The assays were run m quadruplicate and repeated with three different batches of HMVECs.

### Example 2

### Human Dermal Microvascular Endothelial Cells Consist of Distinct Populations of Blood Vascular and Lymphatic Endothelial Cells

The functions of different VEGF receptors have been extensively studied in transfected cell lines, but the lack of an appropriate cellular background can compromise results obtained from such studies. Therefore, the inventors set out to separate microvascular endothelial cells into specific constituent populations of endothelial cells of one type which were substantially free of other types of endothelial cells. More particularly, the inventors generated populations of lymphatic endothelial cells that were substantially free of blood vascular endothelial cells and vice versa. In order to pursue this endeavor and to elucidate VEGFR-3 signaling pathways promoting endothelial cell survival, the inventors used primary endothelial cells, human dermal microvascular endothelial cells (HMVEC) and human umbilical vein endothelial cells (HUVEC). It was determined that all three VEGF tyrosine kinase receptors and the neuropilin-1 co-receptor were expressed in cultures of both HMVE and HUVE cells. These observation are explained in further detail in the present Example.

To examine expression of VEGFR mRNAs in primary human dermal microvascular endothelial cells (HMVEC), human umbilical vein endothelial cells (HUVEC) and in the porcine aortic endothelial (PAE) cell line, a Northern blot containing 8 μg of the mRNAs was probed with radiolabeled cDNA fragments of human VEGF receptors and with b-actin for the control of equal loading. Numbers to the right denote the sizes of the transcripts (kb). VEGFR-3 mRNA expression was stronger in microvascular endothelial cells and therefore these cells were used for the study of VEGFR-3 signaling in subsequent experiments.

Using immunofluorescence, the inventors demonstrated that HMVE cells consist of two distinct populations of blood vascular and lymphatic endothelial cells. Briefly, the immunofluorescence double-staining was performed using antibodies against VEGFR-3 and LYVE-1 with counterstaining of the nuclei by Hoechst fluorochrome. The immunofluorescence staining showed that LYVE-1 expression is not detected in all VEGFR-3 positive cells while some VEGFR-3 negative cells are also weakly stained with LYVE-1 antibodies. Immunolabeling with antibodies against podoplanin, vWF and CD31 also was performed and the nuclei were again stained with the Hoechst fluorochrome. This set of double staining studies revealed that vWF expression occurs primarily in the podoplanin negative cells but weak expression is also detected on podoplanin positive cells. The staining for VEGFR-3, LYVE-1, the counterstaining of the nuclei by Hoechst fluorochrome and the staining for podoplanin were performed using live cells on ice whereas the stains for vWF and CD31 were performed after PFA fixation.

Thus, the immunofluorescence microscopy showed that only a subset of the HMVE cells was positive for VEGFR-3. Antigen-blocking experiments and use of three different monoclonal antibodies indicated that the VEGFR-3 staining was specific. The VEGFR-3 expressing cells grew in distinct islands surrounded by VEGFR-3 negative cells. Based on the previous immunostaining results from human tissues (Jussila et al., Cancer Res. 58:1599-1604, 1998; Lymboussaki et al., Am. J. Pathol., 153: 395403, 1998), it was assumed that the former represented lymphatic and the latter blood vascular endothelial cells. Most, but not all of these cells and a few of the VEGFR-3 negative cells were stained for the lymphatic endothelial cell marker LYVE-1 (Banerji et al., J. Biol. Chem., 144(4)789-801, 1999). The VEGFR-3 positive cells were also specifically stained for podoplanin, another recently identified lymphatic endothelial marker (Breiteneder-Geleff et al., et al., Am. J. Path., 154(2) 385-394, 1999). Similar results were obtained also in FACS analysis. The vWF antigen was more prominently expressed in the blood vascular endothelial cells which were negative for podoplanin. The pan-endothelial cell marker CD31 was detected in all cells, confirming the absence of contaminating non-endothelial cells. Also, according to a Western blot analysis, VEGFR-1 and VEGFR-2 were detected in both endothelial cell populations. Among the freshly isolated HMVE cells, the proportion of VEGFR-3 positive cells was in general over 50%, decreasing upon repeated subculture.

### Example 3

### Analysis of VEGFR Specific Ligands Used for the Cell Survival Experiments

VEGF is an endothelial cell mitogen which has been also shown to protect endothelial cells from starvation and TNF-a induced apoptosis via activation of VEGFR-2 (Gerber et al., J. Biol Chem., 273:30336-30343, 1998; Spyridopoulos et al., J. Mol. Cell. Cardiol., 29:1321-1330, 1997). The abilities of the different VEGFRs to promote endothelial cell survival were compared by using VEGFR specific VEGFs. The specificities of the growth factors used were determined using a cell survival bioassay. For the bioassay, Ba/F3 pre-B cells were stably transfected with a chimeric receptor containing the extracellular domain of human VEGFR-1, VEGFR-2 or VEGFR-3 fused with the transmebrane and cytoplasmic domains of the mouse erythropoietin receptor.

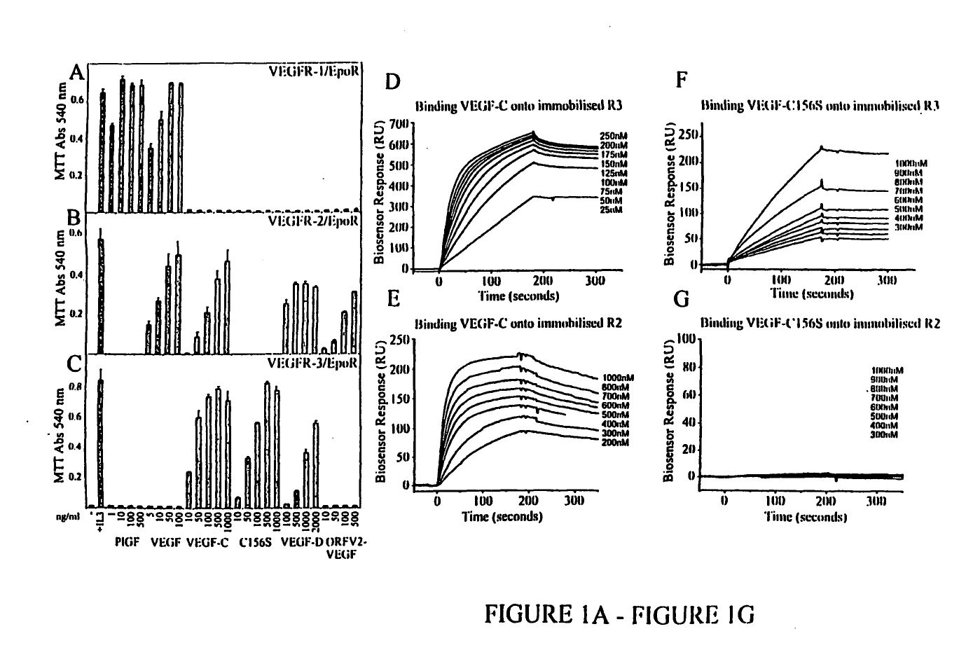

As expected, only VEGF and PIGF were able to induce the survival of VEGFR-1/EpoR cells (FIG. 1A). VEGF, VEGF-C, VEGF-D and orf viral NZ2 (ORFV2-VEGF) were able to support the survival of the VEGFR-2/EpoR expressing cells whereas the mutant VEGF-C156S that binds to and activates only VEGFR-3 (Joukov et al., EMBO J., 15:290-298, 1998) did not affect the survival of these cells (FIG. 1B). Instead, VEGFR-3/EpoR expressing cells survived in the presence of VEGF-C, VEGF-C56S and VEGF-D (FIG. 1C). On the basis of these experiments, VEGF-C concentration of 100 ng/ml and VEGF-C156S concentration of 500 ng/ml, which gave maximal viability in VEGFR-3/EpoR cell survival assays, were chosen for the subsequent apoptosis and signaling experiments.

Biosensor analysis was used to further investigate the interactions of VEGF-C and VEGF-C156S with VEGFR-2 and VEGFR-3. Analysis of the biosensor binding curves confirmed that VEGF-C156S binds only to the extracellular domain of VEGFR-3, whereas wild type VEGF-C bound to both VEGFR-2 and VEGFR-3 receptors (FIG. 1D-FIG. 1G). The analysis of the kinetics of the VEGF-C VEGFR interactions (Table I) revealed lower KD values than reported previously using radioactive ligand binding analysis in cultured receptor expressing cells (Joukov et al., EMBO J., 16:3898-3911, 1997). However, in both assays the affinity of VEGF-C was higher towards VEGFR-3 than towards VEGFR-2. When compared to VEGF-C, the affinity of VEGF-C156S to VEGFR-3 was significantly lower, but of similar magnitude as reported for the interaction of mouse VEGF-D with mouse VEGFR-3 (Baldwin et al., J. Biol. Chem., 276:19166-19171, 2001).

TABLE I
Kinetic data derived from the biosensor analysis of the interaction of
VEGF-C and VEGF-C156S with VEGFR-2 and VEGFR-3. The data
were extracted by global fitting using BIAevaluation 3.0 assuming a
1:1 Langmuirian model with mass transfer.

| Ligand | Receptor | Kₐ(1/Ms) | K_{d}(1/s) | K_{D}(M) |
|---|---|---|---|---|
| hVEGF-C | hVEGFR-2 | 5.5 × 10⁴ | 12.3 × 10⁻⁴ | 2.2 × 10⁻⁸ |
| hVEGF-C | hVEGFR-3 | 13.6 × 10⁴ | 6.05 × 10⁻⁴ | 0.44 × 10⁻⁸ |
| hVEGF-C₁₅₆S | hVEGFR-2 | no binding | no binding | no binding |
| hVEGF-C₁₅₆S | hVEGFR-3 | 0.35 × 10⁴ | 4.0 × 10⁻⁴ | 11.5 × 10⁻⁸ |
| hVEGF-D* | hVEGFR-2 | 1.3 × 10⁴ | 6.3 × 10⁻⁴ | 4.8 × 10⁻⁸ |
| hVEGF-D* | hVEGFR-3 | 1.8 × 10⁴ | 12 × 10⁻⁴ | 6.5 × 10⁻⁸ |
| mVEGF-D* | mVEGFR-2 | no binding | no binding | no binding |
| mVEGF-D* | mVEGFR-3 | 0.8 × 10⁴ | 7.0 × 10⁻⁴ | 8.9 × 10⁻⁸ |

*= reference: Baldwin et al., J. Biol. Chem., 276:19166-19171, 2001
Abbreviations: h = human; m = mouse

The ligands used in the study by Baldwin et al., are the mature forms of VEGF-D, as are VEGF-C and VEGF-C156S used in this study. All other receptors used were bivalent immunoglobulin fusion proteins except mVEGFR-2 which was monovalent.

### Example 4

### VEGFR-3 Signaling Protects Endothelial Cells from Serum Starvation-induced Apoptosis

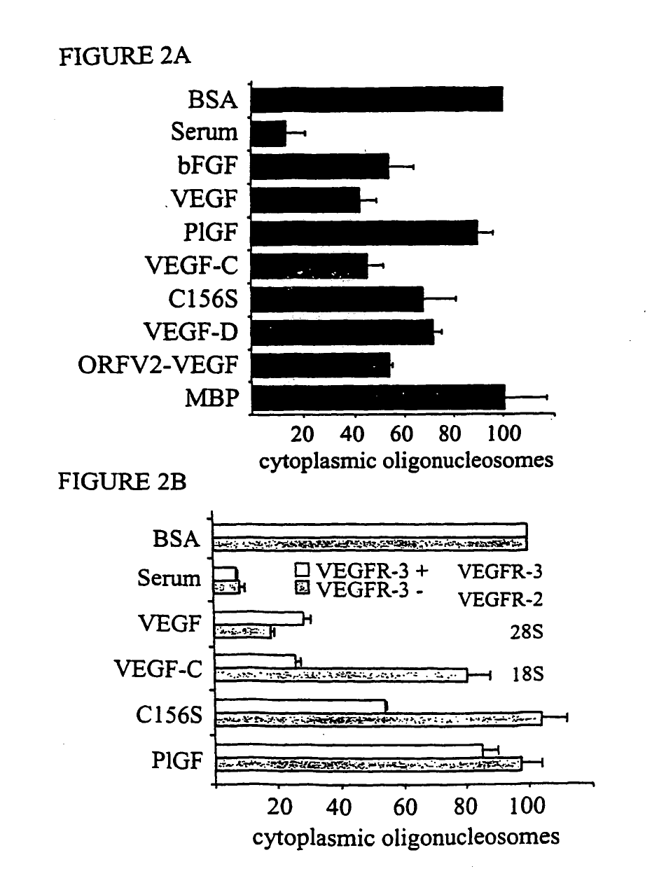

All VEGFs capable of simulating VEGFR-2 or VEGFR-3, or both, including VEGF, VEGF-C, VEGF-C56S, VEGF-D and ORFV2-VEGF, were able to protect microvascular endothelial cells from starvation induced DNA degradation, which was measured as the amount of cytoplasmic histone-associated DNA fragments (FIG. 2A). In contast, PlGF, which binds only to VEGFR-1, did not give significant protection. The lack of VEGFR-1 mediated survival signals was also suggested by the fact that the VEGFR-2 specific ligand, ORFV2-VEGF, gave nearly comparable protection to that obtained with VEGF. VEGF-C and VEGF-C156S inhibited dose-dependently the accumulation of oligo- and mononucleosomes into the serum-deprived lymphatic endothelial cells. The maximum effect of VEGFC was achieved at 100 ng/ml and that of VEGF-C156S at 500 ng/ml.

### Example 5

### Isolation of the VEGFR-3 Expressing Lymphatic Endothelial Cells

In order to compare the effects of the VEGFs on the survival of lymphatic versus blood vascular endothelial cells, specific antibodies and magnetic microbeads were used to isolate and to culture the VEGFR-3 positive and negative cells. Briefly, in this protocol, three separate sets of VEGFR-3 expressing lymphatic endothelial cells were cultured: a first set was cultured in complete medium containing 5% serum, the second set was cultured in complete medium containing 5% serum and supplemented with VEGF (10 ng/ml,) and the third set was cultured in complete medium containing 5% serum supplemented with VEGF-C (100 ng/ml). The VEGFR-3 positive cells were grown for five days after sorting in serum or supplemented with VEGF-C and then stained for podoplanin and proliferating cell nuclear antigen (PCNA). The nuclei were stained with the Hoechst fluorochrome. If supplemented with VEGF-C or VEGF, the cells were stained for PCNA. Immunofluorescence double-staining of non-sorted cells or VEGFR-3 negative and VEGFR-3 positive cell populations with antibodies against podoplanin or VEGFR-3 also was performed.

The lymphatic endothelial cell cultures were over 95% pure according to immunofluorescence staining. The isolated VEGFR-3 positive cells did not adhere well on culture dishes and only few cells proliferated in the complete culture medium containing serum. However, if supplemented with either VEGF or VEGF-C, most of the podoplanin positive cells proliferated readily. In contrast, the blood vascular endothelial cells grew well without the addition of these factors.

The morphology of the isolated lymphatic endothelial cells was more elongated and the cells displayed several protrusions especially when cultured in the presence of VEGF-C. Immunofluorescence for podoplanin and VEGFR-3 colocalized to the same cells in non-sorted, VEGFR-3 negative and VEGFR-3 positive cell populations. In VEGF-C supplemented cultures, only cytoplasmic staining for VEGFR-3 was observed, consistent with internalization of the ligand-receptor complexes. In contrast, in the presence of serum or VEGF, VEGFR-3 was distributed on the cell surface.

### Example 6

### VEGF-C Promotes Survival of Mainly the VEGFR-3 Expressing Lymphatic Endothelial Cells

The accumulation of cytoplasmic mono- and oligonucleosomes was measured as a sign of apoptosis in the two endothelial cell populations during serum starvation. In the VEGFR-3 expressing cells, both VEGF-C and VEGF promoted cell survival (FIG. 2B). However, for the VEGFR-3 negative cells VEGF-C was a less efficient survival factor, requiring five to tenfold higher concentrations for an equal effect as detected in VEGFR-3 positive cells. As expected, VEGF-C156S induced the survival of only the VEGFR-3 positive cells (FIG. 2B). These results confirmed that VEGFR-3 alone can transduce endothelial cell survival signals and that VEGF and VEGF-C target differentially blood vascular and lymphatic endothelial cells. The ability to selectively promote the growth of lymphatic endothelial cell population permits further enrichment of the cell culture for these types of cells.

Serum-deprivation induced apoptosis was also monitored by analyzing the exposure of phosphatidylserine at the cell surface using the fluorescence conjugated phospholipidbinding protein, Annexin-V. AnnexinV stained cells were detected after 24 hours of serum-starvation and by 72 hours of starvation, approximately 40% of the adherent cells were apoptotic, although the cells were more resistant to apoptosis in early passage and at confluence.

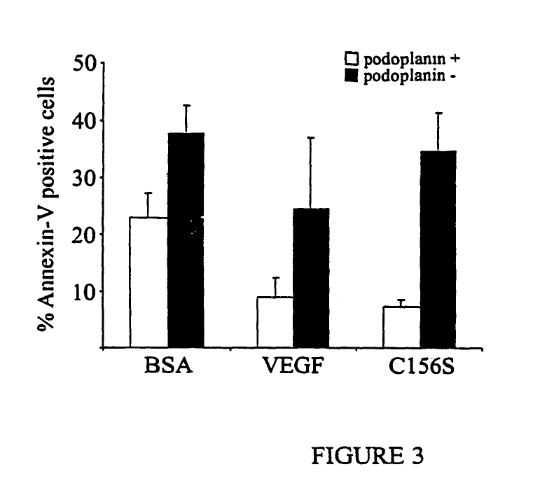

Addition of VEGF to the starvation medium strongly decreased the number of cells displaying Annexin-V positivity as well as cell detachment. Annexin-V staining of the HMVE cells was performed after 72 hours of culture in serum-free medium alone (BSA) or with stimulation of VEGF or VEGF-C. Simultaneous staining using antibodies against podoplanin was used to distinguish lymphatic and blood vascular endothelial cells. Using this staining protocol, it was possible to detect apoptotic, Annexin-V positive lymphatic endothelial cells and apoptotic blood vascular endothelial cells. Again, the nuclei were counterstained with Hoechst fluorochrome. The Annexin-V positive cells were not stained with propidium iodide and thus they represented apoptotic, not necrotic cells. On the other hand, staining of the nuclei by the Hoechst fluorochrome revealed pyknotic nuclei typical for cells undergoing apoptosis. These pyknotic nuclei were also positive for TUNEL staining. Interestingly, stimulation with VEGF-C and especially with VEGF-C156S increased the survival of mainly the lymphatic endothelial cells (FIG. 3). The number of Annexin-V positive cells was higher among the podoplanin negative cells also in the BSA and VEGF treated cultures (FIG. 3). This may be partly an indirect effect, since blood vascular, but not lymphatic endothelial cells produce VEGF-C and therefore they can probably promote the survival of the lymphatic endothelial cells.

### Example 7

### VEGFR-3 Phosphorylation Leads to PI-3kinase Dependent Akt Activation

As discussed in the background, a major signal transduction pathway by which growth factor receptors can promote cell survival employs the PI-3-kinase and its downstream target, the serine-threonine kinase Akt. The effect of the different VEGFs on Akt was analyzed by assessing Akt phosphorylation in serine 473 and threonine 308 using phosphospecific antibodies. Akt was found to be phosphorylated at Ser473 in HMVE cells stimulated by VEGF, ORFV2-VEGF, VEGF-C, VEGF-C156S or VEGF-D, but not in PlGF stimulated HMVE cells. This indicated that Akt is activated by growth factor signals transduced via VEGFR-2 or VEGFR-3, but not via VEGFR-1. A similar increase in Akt Thr308 phosphorylation was also detected. The PI3-kinase inhibitors wortmannin (30 nM) and LY294002 (20 μM) abolished the Akt phosphorylation in response to all the VEGFs studied, demonstrating that the VEGFR-3 mediated Akt activation is transduced via the PI3-kinase as has been previously shown for VEGFR-2 (Gerber et al., J. Biol Chem., 273:30336-30343, 1998; Thakker et al., J. Biol. Chem., 274:10002-10007, 1999).

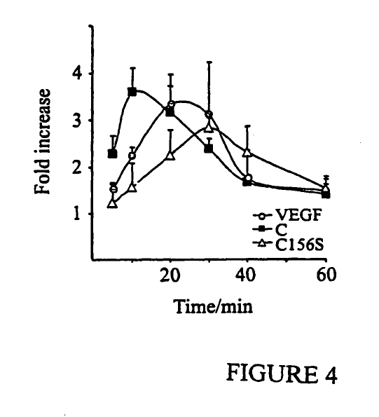

Akt was found to be maximally phosphorylated at 20-30 min after the exposure of HMVEC to VEGF, while the VEGF-C induced Akt phosphorylation peaked at 10 min (FIG. 4). In a striking contrast, VEGF-C156S stimulation resulted in slower Akt phosphorylation, peaking at 3040 min. The differences in the activation of downs targets suggested that Akt phosphorylation via VEGFR-2 or VEGFR-3 may be trasduced via different routes. VEGFR-2 can probably transduce signals for Akt phosphorylation via the classical pathway as it constitutively associates with the regulatory p85 subunit of the PI3-kinase (Thakker et al., J. Biol. Chem., 274:10002-10007, 1999). In contrast, the inventors and others have not been able to detect association of p85 with VEGFR-3 or stimulation of PI3-kinase activity after VEGFR-3 autophosphorylation (Borg el al., Oncogene, 10:973-984, 1995; Pajusola et al., Oncogene, 9:3545-3555, 1994).

### Example 8

### Simultaneous VEGFR-2 and VEGFR-3 Stimulation by VEGF-C Induces a Sustained p42/p44 MAPK Activation

The inventors demonstrated that simultaneous signaling via VEGFR-2 and VEGFR-3 upon VEGF-C stimulation leads to sustained p42/p44 MAPK activation in the HMVE cells. The p42/p44 MAPK activation was detected by Western blotting using phospho-Thr202/Tyr204MAPK specific antibodies and CREB phosphorylation using phospho-Ser133 specific antibodies. The growth factor concentrations used were: VEGF 10 ng/ml, VEGF-C 100 ng/ml and VEGF-C156S 500 ng/ml. Additionally, the present studies showed that VEGFR-3 induced p42/p44 MAPK activation is mediated via protein kinase C in HMVE cells. Effects of inhibition of protein kinase C by GF109203X, MEK1 by PD98059 and PI-3 kinase by LY294002 on p42/p44 MAPK Thr202/Tyr204 phosphorylation, CREB Ser133 phosphorylation and Akt Ser473 phosphorylation in HMVE cells. The growth factor concentrations used were: VEGF 1 ng/ml, VEGF-C 10 ng/ml and VEGF-C156S 500 ng/ml

The mitogen-activated protein kinase (MAPK) signaling pathway is another mechanism implicated in growth factor-dependent cell survival. The inventors demonstrated that simultaneous signaling via VEGFR-2 and VEGFR-3 upon VEGF-C stimulation leads to sustained p42/p44 MAPK activation in the HMVE cells. The p42/p44 MAPK activation was detected by Western blotting using phospho-Thr202/Tyr204-MAPK specific antibodies and CREB phosphorylation using phospho-Ser133 specific antibodies.

The inventors showed that MAPK activation in HMVE cells was detected after VEGFR-3 stimulation by VEGF-C156S. However, MAPK phosphorylation induced by the VEGFR-2 ligands VEGF and VEGFC was significantly stronger in these cells. Although MAPK activation after both VEGF-C and VEGF stimulation peaked at 10-20 min, the VEGF induced activation was more transient than that induced by VEGF-C, which persisted for at least 6 h.

Downstream of the MAP kinases, the MAPK activated kinases, Rsks, have been shown to phosphorylate the transcription factor CREB (cAMP response element-binding protein) at Ser133, which promotes cell survival by increasing transcription of pro-survival genes (Bonni et al., Science, 286:1358-1362, 1999). CREB phosphorylation, which correlated with p42/p44 activation, was detected after stimulation of the HMVECs by VEGF or VEGF-C, but not by VEGF-C156S. Although also Akt has been shown to phosphorylate CREB (Du and Montminy, J. Biol. Chem., 273:32377-32379 1998), inhibition of Akt with LY294002 did not affect CREB phosphorylation. In contrast, inhibition of MEK1 (MAP kinase kinase) with PD98059 or U0126 inhibited VEGF-C, but not VEGF induced CREB phosphorylation.

In addition, the inventors discovered that the VEGFR-3 induced MAPK activation is mediated via PKC. VEGF induced activation of the MAPK cascade has been shown to be mediated by protein kinase C (PKC) instead of the classical Ras pathway (Doanes et al., Biochem. Biophys. Res. Commun. 255: 545-548, 1999; Takahashi et al., Oncogene, 18:2221-2230, 1999; Yoshiji et al., Cancer Res., 59:4413-4418, 1999). In order to study the effect of PKC inhibition on VEGF-C and VEGF-C156S induced MAPK activation, the minimum concentrations of VEGF, VEGF-C and VEGF-C156S were titrated which gave maximal p42/p44 MAPK activation as measured by Western blotting using phosphospecific antibodies. In these conditions, inhibition of PKC by GF109203X completely blocked p42/p44 MAPK phosphorylation induced by VEGF, VEGF-C or VEGF-C156S and CREB phosphorylation induced by VEGF or VEGF-C. Moreover, inhibition of MEK1 by PD98059 resulted in decreased phosphorylation of CREB upon VEGF-C stimulation. Surprisingly, this treatment did not inhibit VEGF induced CREB phosphorylation. In agreement, in a recent study, VEGF induced CREB phosphorylation was shown to be mediated via PKC and p38 MAPK, not via p42/p44 MAPK.

### Example 9

### VEGFR-3 Induces Endothelial Cell Migration

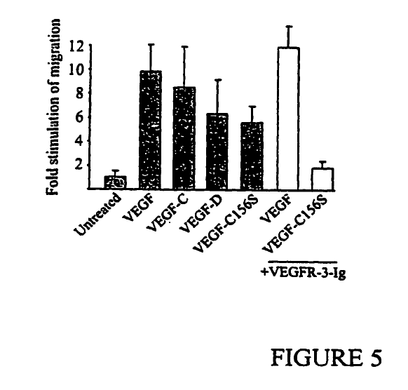

Migration of endothelial cells plays a critical role in angiogenesis and at least some of the VEGF induced migration signals are transduced via PI3-kinase (Gille et al., J. Biol. Chem., 276:3222-3230, 2001; Gille et al., EMBO J., 19:4064-4073, 2000; Qi and Claesson-Welsh, Exp. Cell Res., 263: 173-182, 2001). Since the VEGFR-3 deficient embryos die due to a failure of vascular remodeling (Dumont et al., Science, 282:946-949, 1998), the question of whether VEGFR-3 signaling is also involved in the migration of endothelial cells merited further investigation. The HMVE cells were incubated in the presence of different VEGFs in a Boyden chamber assay. VEGF induced a ten-fold stimulation of cell migration, and the effect of VEGF-C or VEGF-D was nearly comparable to that of VEGF (FIG. 5). Furthermore, VEGF-C156S also induced the migration of HMVE cells, and this was specifically blocked by a ten-fold molar excess of soluble VEGFR-3 (FIG. 5, light grey bar). These results indicated that signaling via VEGFR-3 is sufficient for the induction of endothelial cell migration.

While the compositions and methods of this invention have been described in terns of preferred embodiments, it will be apparent to those of skill in the art that variations may be applied to the processes described herein without departing from the concept, spirit and scope of the invention. All such similar substitutes and modifications apparent to those skilled in the art are deemed to be within the spirit, scope and concept of the invention. Techniques used for the production expression libraries and for the production and isolation of recombinant peptides are well known to those of skill in the art and may be used in conjunction with the present invention.

1. A method for isolating lymphatic endothelial cells from a biological sample comprising lymphatic endothelial cells, the method comprising:
a) contacting said sample with an antibody that preferentially recognizes lymphatic endothelial cells as compared to other endothelial cells, under conditions where the antibody binds lymphatic endothelial cells, and
b) isolating lymphatic endothelial cells bound to said antibody.

2. The method of claim 1, wherein said antibody is an antibody that is immunologically reactive with an epitope on the extracellular domain of VEGFR-3 that is specific for lymphatic endothelial cells.

3. The method of claim 1 wherein said biological sample is from a human patient.

4. The method of claim 1, wherein said antibody is immobilized on a solid support and said biological sample is contacted with said support to allow the lymphatic endothelial cells to become bound to said antibody.

5. The method of claim 1, wherein said antibody is labeled with a fluorescent label and said lymphatic endothelial cells are isolated using fluorescence activated cell sorting.

6. The method of claim 1, wherein said antibody is labeled with a magnetic label and lymphatic endothelial cells are isolated using magnetic activated cell sorting.

7. The method of claim 1, wherein said lymphatic endothelial cells are isolated using immunohistochemistry.

8. The method of claim 1, wherein said lymphatic endothelial cells are isolated using immunochromatography.

9. The method of claim 1, wherein the antibody is a polyclonal antibody.

10. The method of claim 1, wherein the antibody is a monoclonal antibody.

11. The method of claim 1, wherein the antibody is a binding reagent that comprises an antigen binding fragment of 2E11D11.

12. The method of claim 11, wherein the antibody recognizes the same epitope of VEGFR-3 protein that is recognized by 2E11D11.

13. The method of claim 1, wherein said antibody is 2E11D11.

14. The method of claim 1, wherein said antibody is an anti-podoplanin.

15. A method of isolating blood vascular endothelial cells from a sample of microvascular endothelial cells, the method comprising:
a) contacting said cells with an antibody that preferentially binds to lymphatic endothelial cells as compared to other endothelial cells, under conditions where the antibody binds lymphatic endothelial cells, and
b) removing said lymphatic endothelial cells that are bound by said antibody from microvascular cells that are not bound to said antibody, wherein said microvascular cells not bound to said antibody comprise a population of blood vascular endothelial cells substantially free of lymphatic endothelial cells.

16. The method of claim 15, wherein said antibody is an antibody that is immunologically reactive with the extracellular domain of VEGFR-3.

17. A lymphatic endothelial cell population isolated according to a method comprising:
a) contacting a biological sample comprising lymphatic endothelial cells with an antibody that preferentially binds to lymphatic endothelial cells as compared to other endothelial cells, under conditions where the antibody binds lymphatic endothelial cells, and
b) isolating lymphatic endothelial cells that are bound by said antibody.

18. The lymphatic endothelial cell population of claim 17, wherein said antibody is an antibody that is immunologically reactive with the extracellular domain of VEGFR-3.

19. The lymphatic endothelial cell population of claim 17, wherein said biological sample of cells comprises a heterogeneous population of endothelial cells.

20. The lymphatic endothelial cell population of claim 17, wherein said biological sample of cells is a microvascular endothelial cell population.

21. The lymphatic endothelial cell population of claim 17, wherein said lymphatic endothelial cell population is substantially free of contaminating blood vascular endothelial cells.

22. The method of claim 17, comprising expanding said lymphatic endothelial cells.

23. A blood vascular endothelial cell population isolated according to a method comprising:
a) contacting a population of microvascular endothelial cells with an antibody that preferentially binds to lymphatic endothelial cells as compared to blood vascular endothelial cells, under conditions where the antibody binds to lymphatic endothelial cells, and
b) removing said lymphatic endothelial cells that are bound by said antibody from microvascular cells that are not bound to said antibody, wherein said microvascular cells not bound to said antibody comprise a population of blood vascular endothelial cells substantially free of lymphatic endothelial cells.

24. The blood vascular cell population of claim 23, wherein said antibody is an antibody that is immunologically reactive with the extracellular domain of VEGFR-3.

25. The blood vascular cell population of claim 23, wherein the method further comprises expanding said blood vascular endothelial cell population.

26. A lymphatic endothelial cell population substantially free of other contaminating endothelial cells.

27. A blood vascular endothelial cell population substantially free of other contaminating endothelial cells.

28. A method of obtaining a composition substantially enriched in a subpopulation of lymphatic endothelial cells comprising:
(a) obtaining, a source of cells comprising microvascular endothelial cells;
(b) contacting the cells with a monoclonal antibody that preferentially binds to lymphatic endothelial cells as compared to other endothelial cells, under conditions to allow an antibody to bind lymphatic endothelial cells;
(c) separating those cells that are specifically bound by the monoclonal antibody, thereby obtaining a composition substantially enriched in a subpopulation of lymphatic endothelial cells.

29. The method of claim 28, wherein said antibody is an anti-podoplanin antibody.

30. The method of claim 28, wherein said antibody is 2E11D11.

31. A composition comprising a substantially. enriched subpopulation of lymphatic endothelial cells obtained by the method according to claim 28.

32. A method of ameliorating a lymphatic endothelial cell disorder comprising targeting lymphatic endothelial cells with a therapeutic agent, wherein said therapeutic agent is targeted to said cells using an antibody that preferentially binds to lymphatic endothelial cells as compared to other endothelial cells, wherein said antibody is an antibody that is immunologically reactive with the extracellular domain of VEGFR-3.

33. The method of claim 30, wherein said disorder is selected from the group consisting of lymphoma, hereditary lymphedema, lymphedemas, lymphangiomas, lymphangiosarcomas, lymphangiomatosis, lymphangiectasis, and cystic hygroma.

34. A method of ameliorating a lymphatic disorder, wherein said method comprises ex vivo therapy comprising:
a) obtaining microvascular endothelial cells of a patient in need of said therapy;
b) contacting the microvascular endothelial cells with an antibody that preferentially binds to lymphatic endothelial cells as compared to other endothelial cells, under conditions that allow the binding of said antibody to lymphatic endothelial cells;
c) isolating lymphatic endothelial cells that are bound by said antibody
d) transfecting said lymphatic endothelial cells with an expression construct comprising a nucleic acid encoding a therapeutic protein operably linked to a promoter, in an amount effective to produce the expression of said protein in said cells and
e) reintroducing said transfected cells to said patient.

35. The method of claim 34, wherein said antibody is an antibody that is immunologically reactive with the extracellular domain of VEGFR-3.

36. A method of promoting the growth of lymphatic endothelial cells in culture comprising:
a) obtaining the lymphatic endothelial cells according to claim 1;
b) stimulating said cells with a VEGFR-3 ligand;
wherein stimulating the growth of said cells with said VEGFR-3 ligand promotes the survival of said cells in culture as compared to growth in the absence of said stimulation.

37. The method of claim 36, wherein said VEGFR-3 ligand is VEGF-C, VEGF-C156S or VEGF-D.

38. The method of claim 36, further comprising stimulating said cells with a VEGFR-2 ligand.

39. The method of claim 36, wherein said stimulation of said cells protects the cells from apoptosis.

40. The method of claim 36, wherein said protection of said cells is mediated through the activation of Akt or p42/MAPK signaling molecules.

41. The method of claim 36, wherein said stimulation allows said cells to maintain differentiated endothelial cell characteristics.

42. A method of selectively modulating lymphatic endothelial cells in a mammalian organism comprising:
a) isolating lymphatic endothelial cells from said mammalian organism by the method of claim 1,
b) contacting said isolated lymphatic endothelial cells with an agent to modulate the lymphatic endothelial cells; and
c) reintroducing the lymphatic endothelial cells into said organism.

43. The method of claim 42, wherein the contacting step comprises introducing an exogenous polynucleotide into said cells.

44. The method of claim 42, wherein the organism has a disorder characterized by a genetic mutation in a gene expressed in lymphatic endothelial cells and the contacting comprises introducing an exogenous polynucleotide into the cells to overcome the effects of the genetic mutation in said gene.

45. The method of claim 44, wherein said disorder is hereditary lymphedema.

46. A method for imaging lymphatic endothelial cells in tissue from a vertebrate organism, comprising the steps of:
(a) contacting vertebrate tissue suspected of containing a lymphatic endothelial cells with a composition comprising an antibody that preferentially binds to lymphatic endothelial cells as compared to other endothelial cells, under conditions that allow the binding of said antibody to lymphatic endothelial cells;
(b) detecting said antibody bound to said lymphatic endothelial cells in said tissue; and
(c) imaging lymphatic endothelial cells in the tissue by identifying lymphatic endothelial cells bound by said antibody, wherein said binding of the lymphatic endothelial cells to said antibody indicates the presence and location of lymphatic endothelial cells in the tissue.

47. The method of claim 46, wherein said tissue comprises human tissue.

48. The method of claim 46, further comprising the step of washing said tissue, after said contacting step and before said imaging step, under conditions that remove from said tissue antibody that is not bound to the lymphatic endothelial cells in said tissue.

49. The method of claim 46, wherein said antibody is an antibody that is immunologically reactive with the extracellular domain of VEGFR-3.

50. The method of claim 46, wherein said antibody is an anti-podoplanin antibody.

51. The method of claim 46, wherein said antibody further comprises a detectable label covalently bound thereto.

52. The method according to claim 46, further comprising steps of:
contacting the tissue with a second compound that specifically binds to a lymphatic endothelial marker that is substantially absent in blood vascular endothelia; and
detecting said second compound bound to cells in said tissue;
wherein said imaging step comprises identifying lymphatic vessels labeled with both the antibody and the second compound, wherein lymphatic vessels labeled with both the antibody and the second compound correlate with the presence and location of lymphatic endothelial cells in the tissue.

53. The method of claim 52, wherein said antibody is an antibody that is immunologically reactive with the extracellular domain of VEGFR-3, and said second compound is an anti-podoplanin antibody.

54. A method of screening for a disease characterized by a change in lymphatic endothelial cells, comprising the steps of:
(a) obtaining a tissue sample from a vertebrate organism suspected of being in a diseased state characterized by changes in lymphatic endothelial cells;
(b) exposing said tissue sample to a composition comprising an antibody that preferentially binds to lymphatic endothelial cells as compared to other endothelial cells, under conditions that allow the binding of said antibody to lymphatic endothelial cells in said organism;
(c) washing said tissue sample; and
(d) screening for said disease by detecting the presence, quantity, or distribution of said bound antibody in said tissue sample.

55. A method for specifically detecting lymphatic endothelial cells in a mammal, comprising the steps of:
(a) administering to said mammal a composition comprising an antibody that preferentially binds to lymphatic endothelial cells as compared to other endothelial cells, under conditions that allow the binding of said antibody to lymphatic endothelial cells, and
(b) detecting said antibody bound to lymphatic endothelial cells, thereby detecting lymphatic endothelial cells in said organism.

56. The method of claim 55, further comprising administering to said mammal a second compound that specifically binds to a lymphatic endothelial cell marker; and wherein said detecting step comprises detection of said antibody and said second compound bound to lymphatic endothelial cells.

57. A method modifying lymphatic endothelial cells comprising:
a) obtaining a microvascular endothelial cells;
b) contacting the microvascular endothelial cells with an antibody that preferentially binds to lymphatic endothelial cells as compared to other endothelial cells, under conditions that allow the binding of said antibody to lymphatic endothelial cells;
c) isolating lymphatic endothelial cells that are bound by said antibody; and
d) transfecting said lymphatic endothelial cells with an expression construct comprising a nucleic acid encoding a therapeutic protein operably linked to a promoter, in an amount effective to produce the expression of said protein in said cells, wherein said transfecting produces modified lymphatic endothelial cells.

58. A lymphatic endothelial cell produced according to the method of claim 57.

59. A composition comprising a substantially enriched subpopulation of lymphatic endothelial cells obtained by the method according to claim 29.

60. A composition comprising a substantially enriched subpopulation of lymphatic endothelial cells obtained by the method according to claim 30.
